(19) United States
(12) Patent Application Publication
(10) Pub. No.: US 2026/0199216 A1
(43) Pub. Date: Jul. 16, 2026

(54) A RE-DISPERSIBLE BACTERIAL CELLULOSE POWDER

(71) Applicant: Cellugy ApS, Søborg (DK)

(72) Inventors: Alixander Perzon, Søborg (DK); Deby Fapyane, Søborg (DK)

(21) Appl. No.: 19/135,448

(22) PCT Filed: Dec. 7, 2023
(86) PCT No.: PCT/EP2023/084726

§ 371 Date: Jun. 4, 2025

(30) Foreign Application Priority Data
Dec. 7, 2022 (EP) 22211902.6

(51) Int. Cl. A61K 8/73; A61Q 19/00; C08K 5/19; C08L 5/00

(52) U.S. Cl. CPC A61K 8/73; A61K 8/731; A61Q 19/00; C08K 5/19; C08L 5/00; A61K 2800/10

(57) ABSTRACT

The present invention relates to a re-dispersible bacterial cellulose powder and methods of production and uses of the same. The present invention further relates to a bacterial cellulose suspension obtainable from said bacterial cellulose powder and methods of production and uses of the same.

### TECHNICAL FIELD

The present invention relates to a re-dispersible bacterial cellulose powder and methods of production and uses of the same. The present invention further relates to a bacterial cellulose suspension obtainable from said bacterial cellulose powder and methods of production and uses of the same.

### BACKGROUND

In nature cellulose exists in the form of nano-to-millimeter sized fibers as produced by plants or bacteria. Bacterial cellulose (BC) is a source of microfibrillated cellulose fibers (MFC) which is less explored than that of plant-based. BC is obtained in a fermenter usually by growing bacteria of the genus Acetobacter/Gluconobacter/Komagataeibacter. When produced in a dynamic fermenter, the resulting BC consists of diverse cellulosic fibers (from very fine cellulosic fibers with widths of 40 to 100 nm to bigger fibers with lengths of 100-150 μm or longer). Chemically, BC is identical to plant cellulose. However, its morphology aspects such as the high aspect ratio, crystallinity, and purity of BC fibers differentiate this material from plant MFC (plant-based fibers usually contain at least 10-15% impurities such as lignin and hemicelluloses). Due to their unique physical properties, BC fibers tend to form a highly reticulated and fine network structure which results in high viscosity and a strong yield-stress system. Bacterial cellulose has the potential to be an efficient rheology modifier, especially in personal care products due to its non-tacky/non-greasy/non-sticky sensorial properties compared to other natural thickeners. BC is also very effective at suspending particles e.g., decorative micro beads, opacifiers, pearlescents, or encapsulated fragrances. In addition, as crystalline cellulose is highly resistant to harsh environments such as high temperature, extreme pH, salts, and surfactants, using BC in a formulation with aforementioned condition has an advantage over other polymers which are more sensitive to such conditions.

A major drawback of using cellulose fibers as rheology modifier is that BC or plant MFC suspensions are usually supplied as 2-10% suspensions which results in high distribution- and shipping costs, risk of microbial contamination, and difficulty of incorporating the material in certain formulations, as personal care formulators are used in powder form as rheology modifier, which gives freedom in regard to methods of integration in formulas. Generally, if dried as is (pure cellulose dispersed in water), dried fibers will irreversibly aggregate and not re-disperse upon hydration. This is a well-known phenomenon referred to as hornification which involves hydrogen bonding between the fibers. In practise, the effect of drying and re-hydrating cellulose fibers is seen by a drastic loss of viscosity, particle suspending ability, and cellulose-water interaction, compared to their non-dried dried state.

Several approaches have been considered to obtain a dry and re-dispersible BC formulation. Common methods involve drying BC with hydrophilic polymers such as cellulose derivatives (CMC, HEC, HPMC, MC), xanthan gum, and starch, in combination with sugars such as sucrose, saccharose, xylitol, erythritol, or maltodextrin (WO 2006/127810, WO 2020/136629 A1 and WO2023/06733 A1). Other methods involve drying BC with calcium carbonate and glycerine (JP 2873927 B2) or organic solvents (WO 2001/005838 A1). The addition of hydrophilic polymers to BC before drying improves hydration of the cellulose network and an additional agent such as sucrose act as a spacer that prevents fiber bonding. Nonetheless, a large quantity of these ingredients is required: for example, 2-3 different ingredients which in total constitutes 50-500% of the dry weight of the final BC formulation may be required for optimal effect (WO2023/06733 A1). This leads to a dilution in performance of the material in terms of sensorial properties, viscosity, solids stabilization, and stability (pH, temperature, salts, surfactants etc.) in various systems. It was also shown that not all hydrophilic polymers may not work the same way. It was demonstrated that when commercial pectin (complex hydrophilic polysaccharides) was added to wood MFC (which do not naturally contain pectin) in attempt to prevent fibers aggregation and hornification, the resulting powder could not be re-dispersed (Hiasa et al. Journal of Fiber Science and Technology 72 (1): 17-26 (2016), (Hietala et al. Cellulose, 24 (5): 2177-2189 (2017).

Moreover, to re-disperse the powder, high/low shear homogenization with a Silverson mixer or other specialized equipment is required. As such equipment is not readily available and/or not commonly used for certain formulations and compositions and entails a high energy consumption, this limits the potential use of the dry BC formulation e.g. in large scale formulation or do-it-yourself formulations (such as for example a powder to suspension/emulsion prepared at home by the customer by shaking by hand).

### SUMMARY

The inventors of the present invention have discovered how the addition of a small amount of commercial pectin with high galacturonic acid content (i.e. mainly homogalacturonan, HG) prevents bonding between BC fibers upon drying, resulting in a BC powder with excellent re-dispersibility or resuspendability.

The low amount of HG required (e.g. 10-20% of the total dry weight) results in no compromise in performance of the BC formulation in terms of stability (toward pH, temperature, salts, and surfactants), particle suspending ability, and sensorial properties. Also, in contrast to using cellulose derivates which are considered semi-synthetic, HG is completely natural. By drying the BC-HG mixture, a sufficiently fine powder is formed which can be dispersed without the need for high/low-shear homogenization or other specialized mixing equipment. Instead, the powder may be dispersed by more conventional mixing techniques such as propeller or shaking.

Upon resuspension, the powder results in a stable BC suspension that behaves identically to the original non-dried suspension in terms of properties and performance. The powder form enables the BC to be used as a viscosity builder with particle stabilizing properties with standard formulation techniques in many applications such as, but not limited to, personal care, cosmetics, pharmaceutical products, biomedical products, and food products. It further enables improved logistics and reduced shipping costs, eliminates risk of microbial contamination, and facilitates formulation on large scale.

Thus, provided herein is a bacterial cellulose (BC) powder comprising:
a. BC; and
b. homogalacturonan (HG);
   wherein the ratio of HG: BC is at least 0.02:1 (wt:wt).

Further provided herein is a method for producing a BC powder, said method comprising providing a BC suspension and:
b. processing said BC suspension;
c. recovering said BC suspension;
d. mixing said BC suspension with HG, thereby obtaining a BC-HG suspension;
e. drying the BC-HG suspension;
   thereby obtaining a BC powder
wherein the ratio of HG: BC of said BC powder is at least 0.02:1 (wt:wt).

Also provided herein is a method for producing a BC powder, said method comprising the steps of:
a. incubating a cellulose-producing bacteria in a culture medium, thereby obtaining a fermentation broth comprising a BC suspension;
b. processing said BC suspension;
c. recovering said BC suspension;
d. mixing said BC suspension with HG, thereby obtaining a BC-HG suspension;
e. drying the BC-HG suspension;
   thereby obtaining a BC powder,
   wherein the ratio of HG: BC of said BC powder is at least 0.02:1 (wt:wt).

Further provided herein is a BC suspension comprising the BC powder as disclosed herein resuspended in an aqueous solution.

Also provided herein is a BC suspension obtainable by mixing the BC powder as disclosed herein with an aqueous solution.

Further provided herein is a method for preparing a BC suspension, said method comprising mixing the BC powder disclosed herein with an aqueous solution, thereby obtaining a BC suspension.

Also provided herein is the use of the BC powder disclosed herein, or the BC suspension prepared from said BC powder, as a thickener, stabilizer, emulsifier, co-emulsifier and/or rheology modifier.

Further provided herein is the use of the BC powder disclosed herein, or the BC suspension prepared from said BC powder, as a film-forming agent, sensorial enhancer,

SPF-boosting agent and/or anti-wrinkle agent.

Also provided herein is the use of the BC powder disclosed herein, or the BC suspension prepared from said BC powder, as a reinforcer material.

Further provided herein is a composition comprising:
a. the BC powder disclosed herein; or
b. the BC suspension prepared from the BC powder disclosed herein.

Also provided herein is composition a comprising:
a. the BC powder disclosed herein;
b. water; and
c. a compound selected from the group consisting of surfactant, emulsifier, salt, buffer, particles, thickener, stabilizer, glycerine and preservative.

Further provided herein is a composition comprising:
a. the BC powder disclosed herein;
b. water; and
c. a quaternary ammonium compound (QUAT), such as a polyquat;
   optionally wherein the concentration of BC powder is between 0.05 and 3 wt %, for example between 0.1 and 1 wt %, further optionally wherein the concentration of QUAT is between 2 and 20 wt %, such as between 5 and 15 wt %.

Also provided herein is a composition comprising:
a. 0.01 to 3 wt % of resuspended BC powder, wherein the BC powder is the BC powder disclosed herein;
b. 60 to 90 wt % water;
c. 1 to 10 wt % glycerine;
d. 5 to 20 wt % surfactant; and
e. 0.1 to 1% preservative;
   optionally wherein the composition further comprises buffer.

Further provided herein is a product comprising the BC powder as disclosed herein; the BC suspension prepared from said BC powder; or a composition comprising said BC powder or said BC suspension.

### DESCRIPTION OF DRAWINGS

FIG. 1. Illustration of relevant steps for making non-modified BC to powder or modified BC to powder, which are suitable examples of BC powder compositions.

FIG. 2. Oven-dried and milled BC powder compared to spray-dried BC powder obtained from lab or pilot-scale drying.

FIG. 3. Shear thinning profile of “Never dried” BC suspension and “Re-dispersed” BC powder (to suspension) containing 1:0.1 BC:HG ratio.

FIG. 4. Storage and loss modulus profiles (elasticity/gel strength) of powder with BC:HG ratio of 1:0.1 measured as “Never dried” and “Redispersed” BC powder (to suspension)

FIG. 5. 1.6% BC suspensions never dried (left) and re-dispersed BC powder to suspension (right), both with BC:HG ratio of 1:1.

FIG. 6. 0.5% BC suspensions never dried (left) and re-dispersed BC powder to suspension (right), both with BC:HG ratio of 1:0.1.

FIG. 7. Picture of never dried suspension and re-dispersed 0.5% BC powder to suspension obtained with different re-dispersion techniques; 250-650 μm particles were added for particle stabilization capability test.

FIG. 8. Viscosity of never dried suspension and re-dispersed 0.5% BC powder to suspension obtained with different re-dispersion techniques.

FIG. 9. Viscosity of 0.5 and 1% re-dispersed BC powder to suspension at different shear rates at 25° C. measured using rheometer.

FIG. 10. Viscosity at different concentrations of re-dispersed BC powder to suspensions, measured at a shear rate of 1 s⁻¹ at 25° C. using rheometer.

FIG. 11. 0.05% BC suspensions (never dried and re-dispersed powder) shown to be able to stabilize jojoba beads where different BC:HG ratios shows unchanged properties of non-dried and re-dispersed powder to suspension.

FIG. 12. Viscosity of 0.5% re-dispersed BC powder to suspension at different pH measured at a shear rate of 1 s⁻¹ at 25° C. using a rheometer.

FIG. 13. Viscosity of 0.5% re-dispersed BC powder to suspension in presence of different salts and surfactants measured at a shear rate of 1 s⁻¹ at 25° C. using a rheometer.

FIG. 14. Cleanser formulas viscosity containing different amount of re-dispersed BC powder; with glycerine, anionic surfactant (capryl glucoside), amphoteric surfactant (cocamidopropyl betaine), adjusted to pH 5 and 6 showing overall formula stability and viscosity building measured at a shear rate of 1 s⁻¹ at 25° C. using a rheometer.

### DETAILED DESCRIPTION

### Definitions

Bacterial cellulose—The term bacterial cellulose or BC as used herein refers to a material comprising cellulose produced by bacteria. Bacteria producing cellulose include, but are not limited to, bacteria of the genus Acetobacter, Gluconobacter and Komagataeibacter, BC producing bacteria can be used as single strains or combination, such as with each other and/or with other microorganisms, such as other bacteria, yeast and/or fungi. BC, unlike most plant cellulose, does not contain hemicellulose or lignin. Bacterial cellulose can be produced in the form of nano- and/or microfibrillated cellulose.

Circularity—The term circularity as used herein refers to a measure of the similarity of a particle to a circle, particularly a BC powder particle. A circular particle has a circularity value near one. The average circularity may be determined based on the powder particle volume distribution or on the powder particle number distribution using a particle characterization tool, such as a Malvern Morphology G3 microscope as described in Example 7 of the present application.

Clumping—The term clumping as used herein refers to the tendency of particles or substances to gather in clumps, i.e. clusters or aggregates, due to attractive forces between them. This can be seen in various phenomena such as the formation of clusters of molecules in a liquid, or the aggregation of particles in a colloidal solution. In particular, the term clumping as used herein refers to the tendency of cellulose fibers or clusters to form aggregates (clumps) and/or separate from water and/or a water-containing formula.

Coagulation—The term coagulation as used herein refers to the process of suspended cellulose fibers coming together and forming larger aggregates or clumps due to attractive forces between them. This may lead to the formation of a denser and more cohesive structure within the BC suspension. Coagulation in a BC suspension may impact its rheological properties, stability, and overall behaviour due to a shift of fiber structure within the suspension.

Comminution—The term comminution relates to the reduction of solid materials from one average particle size to a smaller particle size, by crushing, grinding, cutting, vibrating or other processes. Comminution may for example be used for preparing a BC suspension from a BC material, whereby the BC material is comminuted into smaller pieces, i.e. into fiber clusters (also called particles, bundles or agglomerates).

Convexity—The term convexity as used herein refers to a particle shape parameter describing the surface properties and the compactness of a particle, in particular a BC powder particle. A particle with smooth surface has a convexity value near one, while a particle with an uneven or rough surface has a lower value. The average convexity may be determined based on the powder particle volume distribution or on the powder particle number distribution using a particle characterization tool, such as a Malvern Morphology G3 microscope as described in Example 7 of the present application.

Crystallinity index (CI)—The crystallinity index, crystallinity or CI, is used to describe the relative amount of crystalline material in cellulose compared to overall crystalline and amorphous regions. The CI is quantitative and is defined as the volume fraction of crystallinity of one phase in a given sample. Methods for measuring the CI are well known in the art. For example, it can be measured using X-ray powder diffraction (XRD), x-ray photoelectron spectroscopy (XPS), solid state 13C NMR, infrared (IR) spectroscopy and Raman spectroscopy. Methods using Fourier transform-IR spectroscopy (FTIR or FT-IR) determine the CI by measuring the relative peak heights or areas from raw spectrographic data. Methods for determining CI using Fourier transform-IR spectroscopy are known in the art (O'Connor et al., 1958). Proteins within a sample can make it difficult to measure the crystallinity and thus determine the CI for example when using FTIR. To circumvent this, the sample can be treated with an alkaline solution, such as but not limited to NaOH and KOH, or enzymes, such as but not limited to alcalase and proteinase, to remove proteins that interfered with the crystallinity measurements e.g. when using FTIR (or other methods) for determining Cl.

Elongation—The term elongation as used herein refers to the length to width ratio of, in particular a BC powder particle. A non-elongated particle has an elongation value near one. The average elongation may be determined based on the powder particle volume distribution or on the powder particle number distribution using a particle characterization tool, such as a Malvern Morphology G3 microscope as described in Example 7 of the present application.

Fiber clusters-fiber clusters, i.e. particles, bundles or agglomerates, also referred to herein as BC fiber clusters and BC clusters, are the solid particles of BC which are present in a BC suspension. The method of production and purification/processing of the BC affects the size of the fiber clusters in the BC suspension. The size of the fiber clusters can be determined by methods known in the art, such as for example by MORFI analysis using a MORFI analyser, such as a MORFI LB01 system. This software performs a discrimination between fibers, shives, and fine elements through size criteria (length and width). Another method to measure fiber cluster size is laser diffraction (LD). LD is a well-known method in the art to analyse the dimensions such as size of particles. LD is based on the diffraction of a laser light/beam when it passes through a particle suspension. The smaller the particle size, the larger the diffraction angle of the laser beam will be. The particle size is calculated using a light scattering model, which can be either Fraunhofer or MIE. The MIE model is more precise for smaller particles (<25 μm) but requires the knowledge of the refractive and absorption index (also known as the real and the imaginary part of the refractive index) of both the sample and the solvent. The precision of the particle size distribution depends on how accurately the optical parameters are known.

Flocculation—The term flocculation or flocculate as used herein refers to a process of contact and adhesion occurring in the BC suspension or a composition comprising the BC suspension, wherein said process results in that the BC particles form larger-size clusters.

Galacturonic acid—The term galacturonic acid or GA as used herein refers to D-galacturonic acid, which in its polymer form polygalacturonic acid (poly-GA) constitutes the main component of homogalacturonan.

High-shear homogenization—The term high shear homogenization as used herein refers to an energy-intensive mixing process which usually involves passage of liquid through a small orifice at high pressure. High shear homogenization may for example be carried out using equipment that by design maximizes shear stress on a suspension or flowing material. High shear homogenization may be carried out using rotor-stator mixers (Ultra-Turrax or Silverson mixer) or high-pressure homogenizers (Microfluidizer or Supermasscolloider).

Homogalacturonan—The term homogalacturonan or HG as used herein refers to a structural component of pectin. HG mainly comprises poly-GA (linear chains of α-(1-4)-linked), with, in some cases, carboxyl groups esterified with e.g. methyl and/or acetyl groups. In one embodiment, HG as used herein refers to a pectin comprising a high content of homogalacturonan, such as at least 70%, such as at least 75%, such as at least 80%, such as at least 85%, such as at least 90%, such as at least 95%, such as at least 99% HG. The amount of HG in pectin or in any other composition, such as in a

BC powder, may be determined using methods well known in the art, such as for example as described by Barnes et al., 2021 or Yapo et al., 2007. HG may also be analysed by degrading the HG to GA and measuring the amount of GA. In one embodiment, HG upon breakdown comprises at least 65% GA, such as at least 70%, such as at least 70%, such as at least 75%, such as at least 80%, such as at least 85%, such as at least 90%, such as at least 95%, such as at least 99%, such as 100% GA.

Low-shear homogenization—The term low shear homogenization as used herein refers to homogenization carried out using mixing equipment such as for example magnetic stirrers, propeller mixers, or simple agitator used at high speed. This imposes lower shear as compared to mixing by high shear homogenization.

Microfibrillated cellulose—The term microfibrillated cellulose (MFC) may refer to a material derived from plants, such as from wood or plant cell walls. MFC may for example be produced by chemical pulping of plant biomass followed by intense mechanical processing of the millimeter sized fibers to break them down into very fine, nanoscale fibrils or microfibrils. Alternatively, MFC may be obtained from bacteria (also known as bacterial cellulose, BC) in the form of fine nano-to-micro scale cellulose fibrils. MFC from bacteria (i.e. BC), unlike most plant cellulose, does not contain hemicellulose or lignin. MFC has unique properties such as a large surface area, reactive hydroxyl groups, high strength-to-weight ratio, high viscosity at low solid content, and high temperature stability.

Particle—The term particle as used herein refers to small or tiny units or pieces of matter, e.g. particles of macroscopic, microscopic or nanoscopic size, e.g. colloidal particles, that may be used in formulations to enhance product performance, texture, and appearance. Such particles may vary in size, composition and properties. Particles may encompass a wide range of materials, from atoms and molecules to larger structures like colloids or aggregates. The particles may serve different purposes, such as providing exfoliation, improving texture, enhancing stability, and delivering active ingredients such as but not limited to actives in the form of oil, microbeads/microspheres, nanoparticles, emollient particles, minerals (fx: clays, silica iron oxide, mica, etc.), pearlescents, colorants (fx: dye, pigments, activated charcoal etx.), polymeric microspheres (fx: starches, gums), adsorbents (fx: activated charcoal, alumina particles), liposomes or nanocapsules (fx; actives such as vitamins or oil embedded in polymer or no-polymeric bodies to enhance absorption to the skin).

Pectin—The term pectin or pectic polysaccharides as used herein refers to a heteropolysaccharide naturally present in the primary cell walls and the green parts of terrestrial plants. Pectin is a macromolecule which consists of three distinct domains: homogalacturonan, rhamnogalacturonan I and rhamnogalacturonan II. Homogalacturonan is a linear homopolymer mainly comprising galacturonic acid (GA). Rhamnogalacturonan I and II comprise a backbone of GA or GA-rhamnose with side chains of neutral sugars. Pectin is produced commercially, and upon its extraction three separate domains listed above (HG, RGI, and RGII) may be obtained. The inventors of the present invention have discovered that mixing bacterial cellulose with pectin having a high content of homogalacturonan and drying said mixture renders a BC powder with excellent re-dispersibility. On the contrary, mixing bacterial cellulose with pectin with a high content of rhamnogalacturonan and drying said mixture renders a BC powder with less suitable re-dispersibility.

Powder—The term powder as used herein refers to a dry solid composed of fine particles (i.e. powder particles) and that may flow freely when shaken or tilted. A powder may have a certain moisture content and still, objectively, be considered a powder. In particular, the term powder as used herein refers to a bacterial cellulose powder as disclosed herein.

Powder particle size—The term powder particle size as used herein refers to the average powder particle size of a powder. The average powder particle size refers in particular to the average circular diameter of the BC powder particles and may be determined using any method known in the art, such as by determining the circular equivalent diameter using a particle characterization tool, such as a Malvern Morphology G3 microscope as described in Example 7 of the present application. Commonly, the average powder particle size is determined based on the powder particle volume distribution or on the powder particle number distribution. The (average) powder particle size based on the number or volume distribution may for example be determined using a particle characterization tool, such as a Malvern Morphology G3 microscope as described in Example 7 of the present application.

Redisperse or redispersion or resuspension—The term redisperse or redispersion or resuspension as used herein refers to the process of redispersing powder particles in a suspension or colloidal system. Various methods may be employed for redispersion, depending on the nature of the particles and the medium. Common redispersion methods include mechanical agitation, ultrasound and high or low shear homogenization/mixing. If a powder is easy to redisperse, redispersion may be for example be carried using low- or zero shear homogenization/mixing. Preferably, powder particles of the easy-to-redisperse powder are evenly distributed throughout the liquid after redispersion. A powder suitable for redispersion may, upon redispersion, for example have the same or similar technological properties and performance as a corresponding never-dried suspension, for example in terms of viscosity and/or stability. A powder suitable for redispersion does preferably not form agglomerates or settle upon redispersion. On the other hand, a powder unsuitable for redispersion may agglomerate or settle when the powder particles are redispersed.

Rhamnogalacturonan—The term rhamnogalacturonan or RG as used herein refers to a structural component of pectin grouped into RG-I and RG-II. RG-I comprises a backbone of repeating residues of GA and α-1,2-L-rhamnose, and side chains with various neutral sugars including but not limited to galactose, arabinose and xylose. RG-II comprises a highly branched backbone of GA with side chains at C-2 and C-3. The side chains include, but are not limited to, arabinose, apiose, fucose, galactose, rhamnose, aceric acid, glucuronic acid, galacturonic acid, xylose and fucose. The amount of RG (i.e. the amount of RG-I and RG-II) in pectin or in any other composition, such as in a BC powder, may be determined using methods well known in the art, such as for example as described by Barnes et al., 2021 or Yapo et al., 2007.

Stable suspension—The term stable suspension or stable BC suspension as used herein refers to a BC suspension which possesses one or more traits that are characteristic for a stable suspension. In other words, a stable suspension may be a suspension which does not flocculate, does not coagulate, does not clump and/or does not exhibit water-cellulose phase separation, for example upon storage for a certain amount of time or when testing said traits according to methods known in the art and disclosed herein. A stable suspension may further be characterized by that the above-mentioned traits are maintained upon extended storage; in high and/or low pH; in the presence of salt, surfactant and/or solvent, such as ethanol. A stable suspension may further be capable of suspending particles in a stable manner, such as that said suspension is capable of suspending particles for a certain amount of time without said particles sedimenting and/or precipitating. A stable BC suspension may for example be prepared from the BC powder disclosed herein.

Suspension—A suspension is a heterogeneous mixture of a fluid which contains solid particles sufficiently large for sedimentation. In other words, it is a heterogeneous mixture in which the solute particles do not dissolve, but get suspended throughout the bulk of the solvent, left floating around freely in the medium.

Viscosity—Viscosity (herein dynamic viscosity), is a measure of the viscosity of a fluid, i.e. a measure of its resistance to deformation at a certain rate. The higher the viscosity, the thicker the fluid; the lower the viscosity, the thinner the fluid. Viscosity can be measured using methods known in the art, such as for example with a rheometer. The viscosity is measured against the shear rate, usually for a certain amount of time and at a specific temperature (such as 25° C.), and has a unit of mPa·s (·10⁻³ kg m⁻¹ s⁻¹) or Pa·s (kg m⁻¹s⁻¹).

Water cellulose separation-Water cellulose separation as used herein refers to the process of BC fibers separating from water in a water-based BC suspension or solution. The process may involve sedimentation (cellulose aggregates to the bottom), flocculation and/or coagulation of the BC fibers.

Water holding capacity—The water holding capacity of a material, such as BC, is the ability of said material to physically hold water during the application of force, pressure, centrifugation and/or heating. The water holding capacity of a BC suspension may be tested using methods well known in the art. For example, the water holding capacity may be determined as described in Example 6 of the present application.

Zero-shear homogenization—The term zero shear homogenization as used herein refers to homogenization which essentially results in no shearing forces being applied to the solution/composition which is being homogenised. Zero shear homogenization may for example be carried out using mixing equipment such as for example a shaker, magnetic stirrers, propeller mixers, deflocculators, or simple agitators used at low speed.

### Bacterial Cellulose Powder

The present invention relates to bacterial cellulose (BC) powder comprising BC and homogalacturonan (HG). The BC powder presented herein is characterized by its excellent re-dispersibility. Upon resuspension, the powder results in a stable BC suspension that behaves identical to its original never-dried suspension in properties and performance, for example in terms of stability, viscosity and function. In other words, the powder is easy to redisperse and does also, upon redispersion, maintain its technological properties and performance as compared to a never-dried BC suspension.

In one aspect, the present invention provides a bacterial cellulose powder comprising:
a. BC; and
b. HG;
   wherein the ratio of HG: BC is at least 0.02:1 (wt:wt).

The ratio of BC:HG may be any ratio suitable for obtaining a BC powder that is fully re-dispersible.

Note that throughout the present disclosure, whenever a ratio is given, the ratio refers to the weight (wt:wt) ratio of the indicated compounds, unless anything else is stated.

In one embodiment, the HG: BC ratio of the BC powder is at least 0.05:1, such as at least 0.1:1, such as at least 0:15:1, such as at least 0.2:1, such as at least 0.5:1, such as at least 1:1.

In one embodiment, the HG: BC ratio of the BC powder is at most 50:1, such as at most 20:1, such as at most 10:1, such as at most 5:1, such as at most 2:1.

In one embodiment, the HG: BC ratio of the BC powder is between 0:02:1 and 50:1, such as between 0.05:1 and 20:1, such as between 0.05:1 and 10:1, such as between 0.1:1 and 10:1, such as between 0.1:1 and 5:1, such as between 0.1:1 and 1:1, such as between 0.1:1 and 0.5:1., such as between 0.1:1 and 0.2:1., such as between 0.05:1 and 0.25:1., such as between 0.1:1 and 1.5:1.

As stated above, the BC powder is characterized by being easy to re-disperse, i.e. to resuspend. In other words, the BC powder when mixed with a solvent generates a homogenous suspension following a period of shaking. The BC powder may for example by resuspended by mixing the sample with a solvent, such as water, and shaking by hand. One additional advantage of the present BC powder is thus that it does not require energy and time intensive mixing processes when redispersing the powder, e.g. in a composition or formulation.

Thus, in one embodiment, the BC powder is re-dispersible in aqueous media by mixing the sample with an aqueous solution and shaking said solution.

In one embodiment, the BC powder is re-dispersible in aqueous media by zero shear or low shear mixing.

In one embodiment, the BC powder is re-dispersible in aqueous media by mixing with a deflocculator, such as a deflocculator mixer, for example a deflocculator turbine.

In one embodiment, the BC powder is re-dispersible in aqueous media by mixing at a very low speed, such as by mixing at at most 100 rpm, such as at at most 200 rpm, such as at at most 300 rpm, such as at at most 400 rpm, such as at at most 500 rpm, such as at at most 600 rpm.

In one embodiment, the BC powder is re-dispersible in aqueous media by mixing at between 200 rpm and 20,000 rpm, such as at between 300 rpm and 20,000 rpm, such as between 500 rpm and 5000 rpm, such as between 600 rpm and 10,000 rpm, such as between 300 rpm and 1000 rpm, such as at between 400 rpm and 800 rpm.

In one embodiment, the BC powder is re-dispersible in aqueous media by mixing for at most 30 seconds, such as for at most 1 minute, such as for at most 2 minutes, such as for at most 5 minutes.

In one embodiment, the BC powder is re-dispersible in aqueous media by mixing for between 30 seconds and 25 minutes, such as by mixing for between 1 and 20 minutes.

In one embodiment, the BC powder is re-dispersible in by mixing at toom temperature, for example at between 20° C. and 25° C.

Furthermore, as stated above, upon resuspension, the powder results in a stable BC suspension having similar or identical properties as the original non-dried suspension, for example in terms of viscosity and water holding capacity.

Thus, in one embodiment, the BC powder has a recovery of viscosity of at least 40% upon redispersion, such as at least 50%, such as at least 60%, such as at least 70%, such as at least 80%, such as at least 90%, such as a recovery of viscosity of 100% upon redispersion,

wherein the recovery of viscosity upon redispersion is relative to the viscosity of a corresponding never-dried BC suspension having the same concentration of BC, and wherein the recovery of viscosity is calculated using the formula:

$$Viscosity recovery ( \% ) = 100 * \frac{Viscosity ( at 1 {s}^{- 1} ) of redispersed BC powder}{Viscosity ( at 1 {s}^{- 1} ) of never dried BC suspension}$$

In one embodiment, the BC powder has a recovery of water holding capacity of at least 50% upon redispersion, such as at least 60%, such as at least 70%, such as at least 80%, such as at least 90%, such as a recovery of water holding capacity of 100% upon redispersion,

wherein the recovery of water holding capacity is relative to the viscosity of a corresponding never-dried BC suspension, and wherein the recovery of viscosity is calculated using the formula:

$$Water holding capacity revovery ( \% ) = 100 * \frac{Water holding capacity of redispersed BC powder}{Qater holding capacity of never dried BC suspension}$$

The BC powder may comprise powder particles with any suitable characteristics and morphological properties, such as any suitable size, such as circular equivalent diameter, convexity, circularity, elongation, shape and/or surface.

In one embodiment, the BC powder comprises powder particles with an average powder particle size of between 0.1 and 500 μm, such as between 1 and 100 μm, such as between 1 and 50 μm, such as between 10 and 40 μm, such as between 15 and 30 μm, such as between 19 and 24 μm, wherein the average powder particle size is calculated based on the powder particle volume distribution.

In one embodiment, the BC powder comprises powder particles with an average powder particle size of between 0.01 and 200 μm, such as between 0.01 and 100 μm, such as between 0.1 and 200 μm, such as between 0.1 and 20 μm, such as between 1 and 15 μm, such as between 1 and 10 μm, such as between 2 and 9 μm, such as between 4 and 8 μm,

wherein the average powder particle size is calculated based on the powder particle number distribution.

In one embodiment, the BC powder comprises powder particles with an average convexity of at least 0.8, such as at least 0.85, such as at least 0.9, such as at least 0.92, such as at least 0.94, such as at least 0.96, such as at least 0.97, wherein the average convexity is calculated based on the powder particle number distribution or based on the powder particle volume distribution.

Thus, in one embodiment, the BC powder comprises powder particles with a smooth surface.

In one embodiment, the BC powder comprises powder particles with an average elongation of between 0.4 and 0.7, such as between 0.45 and 0.65, wherein the average elongation is calculated based on the powder particle volume distribution.

In one embodiment, the BC powder comprises powder particles with an average elongation of between 0.1 and 0.4, such as between 0.2 and 0.3, wherein the average elongation is calculated based on the powder particle number distribution.

Thus, in one embodiment, the BC powder comprises elliptical-shapes powder particles.

In one embodiment, the BC powder comprises powder particles with an average circularity of at least 0.7, such as at least 0.75, such as at least 0.8, such as at least 0.85, such as at least 0.9, wherein the average circularity is calculated based on the powder particle volume distribution.

In one embodiment, the BC powder comprises powder particles with an average circularity of at least 0.7, such as at least 0.75, such as at least 0.8, wherein the average circularity is calculated based on the powder particle number distribution.

Thus, in one embodiment, the BC powder comprises powder particles with a circular shape.

Any of the above-mentioned properties of the BC powder particles may be determined using any suitable method known in the art. For example, the above-mentioned properties of a BC powder particle may be determined by determining the circularity, convexity, elongation and circular equivalent (CE) diameter of the powder particles using a Malvern Morphology G3 microscope as demonstrated in Example 7 of the present application. All of said parameters may be calculated based on the number distribution or based on the volume distribution as also described in Example 7 of the present application.

The BC of the BC powder preferably has a crystallinity that is suitable for obtaining a BC powder that is easily re-dispersible.

In one embodiment, the BC has crystallinity of at least 30%, such as at least 40%, such as at least 50%, such as at least 60%, such as at least 70%, such as at least 80%, when the crystallinity is determined with Fourier Transform Infrared spectroscopy (FT-IR) by calculating the peak ratio at 1430 and 898 cm-1.

In one embodiment, the BC has crystallinity of between 30% and 100%, such as between 50% and 100%, such as between 40% and 90%, such as between 60% and 90%, such as between, 70% and 90%, such as between 70% and 85%, when the crystallinity is determined with FT-IR by calculating the peak ratio at 1430 and 898 cm-1

As stated above, the inventors have discovered that mixing BC with pectin with high content of HG (i.e. high content of GA) and drying said mixture renders a BC powder with excellent re-dispersibility. In other words, the BC powder when mixed with a solvent generates a homogenous suspension following a period of shaking. The BC powder may for example by resuspended by mixing the sample with a solvent, such as water, and shaking by hand.

In one embodiment, the HG is comprised in pectin.

In one embodiment, the pectin comprises at least 65% HG, such as at least 70%, such at least 75%, such as at least 80%, such as at least 85%, such as at least 90%, such as at least 95%, such as at least 99% HG. Pectins comprising such HG contents are known and available to the skilled person, e.g. from Yapo et al., 2007.

HG mainly consists of galacturonic acid. In particular, HG comprises linear chains of α-(1-4)-linked D-galacturonic acid, i.e. polygalacturonic acid.

Analysis of HG content (e.g. in pectin) may thus for example be carried out by degrading the HG to GA, and measuring the GA content.

In one embodiment, the HG upon breakdown comprises at least 65% GA, such as at least 70%, such as at least 70%, such as at least 75%, such as at least 80%, such as at least 85%, such as at least 90%, such as at least 95%, such as at least 99%, such as 100% GA.

In one embodiment, the pectin upon breakdown comprises at least 65% GA, such as at least 70%, such as at least 75%, such as at least 80%, such as at least 85%, such as at least 90%, such as at least 95% GA.

Analysis of HG content (e.g. in pectin) may however also be carried out by other methods known in the art, without degrading the HG to GA. The analysis of HG content may for example be carried out as described by Yapo et al. (2007).

The inventors have further discovered that mixing BC with pectin having a high rhamnogalacturonan (RG) content and drying said mixture renders a BC powder with less suitable re-dispersibility.

Thus, in one embodiment, RG: BC ratio of the BC powder is at most 1:1, such as at most 0.5:1, such as at most 0.1:1, such as at most 0.05:1, such as at most 0.01:1, such as at most 0.005:1.

In one embodiment, the pectin comprises at most 35% RG, such as at most 30% RG, such as at most 25% RG, such as at most 20% RG, such as at most 15%, such as at most 10% RG.

Analysis of RG content (e.g. in pectin) may be carried out by various methods known in the art. The analysis of RG content may for example be carried out as described by Yapo et al. (2007).

There are two types of RG-RG-I and RG-II. RG-I and RG-II have a GA backbone, but mainly consists of various neutral sugars that constitute sides chains of the GA backbone. Hence, RG does to a much lesser extent comprise GA as compared to HG.

In one embodiment, RG upon breakdown comprises less than 65% GA, such as less than 60%, such as less than 55%, such as less than 50%, such as less than 45%, such as less than 40%, such as less than 35%, such as less than 30%, such as less than 20%, such as less than 15% GA.

BC is commonly supplied in the form of microfibrillated cellulose (MFC). Other types of MFCs include cellulose from plants, such as wood, or other sources, such as algae, fungi, and tunicates. BC, unlike wood and some plant celluloses, does not comprise molecules, e.g. lignin or hemicellulose, that may make it difficult to prepare a re-dispersible cellulose powder by mixing the MFC with HG. The high purity, i.e. absence of lignin or hemicellulose, of BC MFC makes it suitable for the applications described herein, in particular for preparing a BC powder as described herein. However, other highly pure microfibrillated celluloses from other sources than bacteria may also be suitable for the applications described herein, in particular for preparing a cellulose-homogalacturonan powder. Other suitable highly pure celluloses include cellulose derived from tunicates, which are natural cellulose producers, as well as MFC from fungi, algae and plant cell walls that are known to be low in non-cellulosic polymers, such as cotton.

Method for producing bacterial cellulose powder The present invention further relates to methods for producing a BC powder, in particular the BC powder described in the section “Bacterial cellulose powder”. Hence, in one embodiment, the present invention relates to a method of producing a BC powder, said method comprising providing a BC suspension and:
b. processing said BC suspension;
c. recovering said BC suspension;
d. mixing said BC suspension with HG, thereby obtaining a BC-HG suspension;
e. drying the BC-HG suspension;
thereby obtaining a BC powder,
   wherein the ratio of HG: BC of said BC powder is at least 0.02:1 (wt:wt).

In one embodiment, the method comprises the steps of:
a. incubating a cellulose-producing bacteria in a culture medium, thereby obtaining a fermentation broth comprising a BC suspension;
b. processing said BC suspension;
c. recovering said BC suspension;
d. mixing said BC suspension with HG, thereby obtaining a BC-HG suspension;
e. drying the BC-HG suspension;
   thereby obtaining a BC powder,
   wherein the ratio of HG: BC of said BC powder is at least 0.02:1 (wt:wt).

The BC powder produced by said method may in particular be as described above in the section “Bacterial cellulose powder”.

The cellulose-producing bacteria used in the method may be any type of bacteria capable of producing cellulose. Preferably, the bacteria are native cellulose producers.

Hence, in one embodiment, the cellulose-producing bacteria is of a genus selected from the group consisting of Acetobacter, Gluconacetobacter, Komagataeibacter and Gluconobacter.

In one embodiment, the cellulose-producing bacteria is of a species selected from the group consisting of Acetobacter xylinus, Acetobacter xylinum, Acetobacter hansenii, Acetobacter pasteurianus, Gluconacetobacter xylinus, Gluconobacter oxydans, Komagataeibacter rhaeticus and Komagataeibacter xylinus. In a preferred embodiment, the cellulose-producing bacteria is Komagataeibacter xylinus.

The cellulose producing bacteria Komagataeibacter xylinus is also known as Acetobacter xylinus and Gluconobacter xylinus. All three names are used herein interchangeably.

The incubation of step a. may take place in any type of reactor suitable for producing bacterial cellulose. Preferably, however, the incubation of step a. takes place in an agitated reactor, such as in a stirred tank reactor. In one embodiment, the incubation of step a. is between one and six days, such as between two and five days.

An advantage of producing the BC suspension of step a. in an agitated reactor is that comminution, for example wet comminution, can be dispensed with, i.e. may not be required during BC suspension processing. Hence, in one embodiment, step b. (i.e. processing said BC suspension) does not comprise a step of wet comminution.

In one embodiment, the culture medium used in step a. is Hestrin-Schramm (HS) medium.

The processing of step b. may comprise one or more steps, as suitable for obtaining a BC suspension suitable for preparing a BC powder as described herein. In one embodiment, the processing of step b. comprises the following sub-steps:
i. washing the recovered BC suspension with water;
ii. treating the BC suspension with an alkali, preferably with sodium hydroxide;
iii. neutralizing the BC suspension with acid; and/or
iv. separating the BC from the fermentation broth, optionally by filtration.

In one embodiment, the mild alkali used in step ii. is sodium hydroxide. In one embodiment, the concentration of sodium hydroxide is at most 10 g/L, such as at most 9 g/L, such as at most 8 g/L, such as at most 7 g/L, such as at most 6 g/L, such as at most 5 g/L, such as at most 4 g/L, such as at most 3 g/L, such as at most 2 g/L, such as at most 1 g/L. In one embodiment, the concentration of sodium hydroxide is between 0.001 and 0.5 M, such as between 0.2 and 0.8 M, such as between 0.1 and 0.4 M, such as between 0.2 and 0.3 M, such as 0.25 M sodium hydroxide.

In one embodiment, the mild alkali used in step ii. is potassium hydroxide.

In one embodiment, step ii. is performed at a temperature of between 5° and 80° C.

In one embodiment, the treatment of step iii. comprises treating the BC until it has a pH of between 2 and 8, such as a pH of between 6 and 7, such as a pH between 4 and 7, such as a pH of between 2 and 4, such as a pH of between 2.5 and 3.5, optionally a pH of 3. In one embodiment, said treatment comprises the use of acid, such as for example HCl, optionally HCl with a concentration between 0.001 and 0.5 M.

In one embodiment, step d. comprises homogenization of the BC-HG suspension.

In one embodiment, step e. comprises or consists of spray drying or oven drying the BC-HG suspension. In a preferred embodiment, step e. comprises spray drying the BC-HG suspension.

Bacterial Cellulose Suspension Produced from Bacterial Cellulose Powder

The BC powder of the present invention has, as stated above, excellent re-dispersibility and may render, upon re-dispersion, a BC suspension with properties identical or close to identical as compared to a never-dried BC suspension.

Thus, in one aspect, the present invention provides a BC suspension comprising the BC powder as described herein in the section “Bacterial cellulose powder”, wherein said BC suspension is obtainable by mixing said BC powder with an aqueous solution.

In one embodiment, the present invention provides a BC suspension comprising the BC powder as described herein in the section “Bacterial cellulose powder” resuspended in an aqueous solution.

The aqueous solution may be, but is not in any way limited to, water.

In one embodiment, the present invention provides a BC suspension wherein the ratio of HG: BC is at least 0.02:1 (wt:wt).

In one embodiment, the HG: BC ratio of the BC suspension is at least 0.05:1, such as at least 0.1:1, such as at least 0:15:1, such as at least 0.2:1, such as at least 0.5:1, such as at least 1:1.

In one embodiment, the HG: BC ratio of the BC suspension is at most 50:1, such as at most 20:1, such as at most 10:1, such as at most 5:1, such as at most 2:1.

In one embodiment, the HG: BC ratio of the BC suspension is between 0:02:1 and 50:1, such as between 0.05:1 and 20:1, such as between 0.05:1 and 10:1, such as between 0.1:1 and 10:1, such as between 0.1:1 and 5:1, such as between 0.1:1 and 1:1, such as between 0.1:1 and 0.5:1., such as between 0.1:1 and 0.2:1., such as between 0.05:1 and 0.25:1., such as between 0.1:1 and 1.5:1.

Thus, in one embodiment, RG: BC ratio of the BC powder is at most 1:1, such as at most 0.5:1, such as at most 0.1:1, such as at most 0.05:1, such as at most 0.01:1, such as at most 0.005:1.

A BC suspension prepared from the BC powder described in the section “Bacterial cellulose powder” possesses several superior properties related to stability. In other words, the BC suspension prepared from the BC powder described in the section “Bacterial cellulose powder” presented herein is particularly stable.

The BC suspension prepared from the BC powder disclosed herein is referred to as “BC suspension” and “BC suspension prepared from BC powder” interchangeably in this section.

In one embodiment, the BC suspension:
does not clump;
does not flocculate; and/or
does not exhibit water-cellulose separation.

In one embodiment, the BC suspension does not clump, does not flocculate and/or does no exhibit water-cellulose separation after at least 3 months of storage, such as after at least 4 months of storage, such as after at least 6 months of storage, such as wherein said suspension does not exhibit water-cellulose separation after at least 1 year of storage.

Flocculation, clumping, and coagulation are related terms that describe processes involving the aggregation of particles in a liquid, such BC fibers in a BC suspension.

Flocculation refers to the process in which small particles in a liquid come together to form larger aggregates called flocs or floccules. These flocs are larger and more easily settleable than individual particles.

Clumping is the gathering together of particles, molecules, or substances into clusters or groups. Clumping can occur due to various forces, such as attractive forces between particles or external influences like temperature changes or chemical reactions.

Coagulation is a specific type of flocculation that involves the formation of larger, insoluble particles from smaller suspended particles in a liquid. Coagulation may occur as a result of a chemical reaction.

In general, flocculation, clumping and coagulation all result in water-cellulose separation.

Whether or not a BC suspension is stable and does not flocculate, clump, coagulate and/or does not exhibit water-cellulose separation can be tested using methods well known in the art.

Whether or not a BC suspension clumps may for example be tested by applying rotation motion through a rheometer for a few minutes at a shear rate of 100/s. If the BC suspension does not clump, no clumps or aggregates should be visible by the eye after said test. Whether or not a BC suspension clumps may alternatively be tested more qualitatively, by rolling a suitable amount of BC suspension between the fingers. If the BC suspension does not clump, the cellulose should form a small ball and no clumps should be sensed or seen when performing said roll.

Similarly, whether or not a BC suspension exhibits water cellulose separation may for example be tested by applying rotation motion through a rheometer at a shear rate of 100/s. If the BC suspension does not exhibit water cellulose separation, the water and the cellulose should not form separate phases after said test, i.e. no separate phases should be visible by the eye after said test. Alternatively, whether or not a BC suspension exhibits water cellulose separation may for example be tested by storing the BC suspension for a specific amount of time, such as for example for at least one weeks, such as at least two weeks, such as at least four weeks, such as at least one month, such as at least two months, such as at least four months, such as at least six months, such as at least one year. The BC suspension may for example be stored in a refrigerator at 4° C. If the BC suspension does not exhibit water cellulose separation, the water and the cellulose should not form separate phases after said test, i.e. no separate phases should be visible by the eye after said test.

Whether or not a BC suspension coagulates or flocculates may for example be tested by applying rotation motion through a rheometer for a few minutes at a shear rate of 100/s. If the BC suspension does not coagulate or flocculate, no coagulates or flocculates should be visible by the eye after said test. Whether or not a BC suspension coagulates or flocculates may alternatively be tested more qualitatively, by rolling a suitable amount of BC suspension between the fingers. If the BC suspension does not coagulate or flocculate, the cellulose should form a small ball and no coagulates or flocculates should be sensed or seen when performing said roll.

Hence, whether or not a BC suspension is stable may for example be tested by any of the above-mentioned tests of clumping, water-cellulose separation and flocculation. In other words, if the BC suspension does not clump, does not flocculate and/or does not exhibit water-cellulose separation, said BC suspension may be considered stable.

Due to its stability and the above-mentioned properties, the BC suspension produced from the BC powder disclosed herein further has high particle-suspending properties. In other words, said BC suspension is capable of suspending particles in a stable manner, i.e. so that the particles do not settle and/or precipitate after a certain amount of time.

In one embodiment, said particles have a size of between 0.1 mm and 5 mm in diameter, such as between 0.25 mm and 0.65 m, such as between 1 mm and 2 mm, such as between 1 mm and 1.5 mm, such as between 0.25 mm and 2 mm, such as between 0.1 mm and 4 mm, such as between 0.1 mm and 3 mm.

In one embodiment, said particles have a size of no more than 5 mm, such as no more than 4 mm, such as no more than 3 mm, such as no more than 2 mm, such as no more than 1 mm, such as no more than 0.5 mm, such as no more than 0.1 mm, such as no more than 500 μm, such as no more than 100 μm, such as no more than 10 μm, such as no more than 1 μm.

The particles may be selected from the group consisting of pearlescents; decorative beads; fragrance beads; gelatine beads; jojoba beads; polyethylene beads; charcoal particles; apricot kernel particles; colorant particles; pigments; air bubbles; insoluble enzymes; encapsulated actives such as moisturizers or exfoliating agents such as alpha-hydroxy acids and glycolic acid; inorganic minerals; kaolin; zeolites; TiO2; ZnO2; vitamins such as vitamin A or vitamin E; oil droplets; oil suspensions; and oil emulsions. In one embodiment, the particles comprise an active ingredient such as:
i. a vitamin, such as vitamin A or vitamin E;
ii. retinol, or
ii. an active ingredient which is not soluble in water but is suspendable in water or oil.

Whether or not a BC suspension is capable of suspending particles in a stable manner can be tested by suspending particles in the BC suspension, and storing the BC suspension with particles for a certain amount of time. If the particles are still suspended, and have not .e.g. sedimented and/or precipitated, said BC suspension is capable of stably suspending particles.

Yet another advantage is that the BC suspension prepared from the BC powder disclosed herein:
a. is stable;
b. does not clump;
c. does not flocculate;
d. does not exhibit water-cellulose separation;
e. is capable of stabilizing particles; and/or
f. maintains its viscosity;
   in the following conditions:
for at least 6 months of storage, such as for at least 9 months of storage, such as for at least 1 year of storage;
at a pH of between 2 and 12, such as at a pH of between 3 and 11; and/or
at high temperatures.

Even further, the BC suspension prepared from the BC powder disclosed herein:
a. is stable;
b. does not clump;
c. does not flocculate;
d. does not exhibit water-cellulose separation;
e. is capable of stabilizing particles; and/or
f. maintains its viscosity;
   in the presence of one or more of the following compounds:
surfactant;
salt;
oil;
buffer;
particles;
emulsifier;
thickener;
stabilizer; and/or preservative.

The surfactant may for example be a cationic surfactant, a zwitterionic surfactant, an anionic surfactant, an amphoteric surfactant and/or a nonionic surfactant.

In one embodiment, the surfactant is selected from the group consisting of cocoamidpropyl betaine (CAPB), sodium dodecyl sulfate (SDS), sodium laureth sulfate (SLS), lauryl glucoside and surfactants comprising quaternary ammonium cation (QUAT), decyl glucose, polyquaternium-7 (QUAT 7), behentrimonium chloride, glyceryl stearate, sodium cetearyl sulphate, glyceryl caprylate, sorbitan laurate/C18-C20 and glycol isostearate.

In one embodiment, the concentration of surfactant in the BC suspension is at least 5 wt %, such as at least 10 wt %, such as at least 15 wt %, such as at least 20 wt %, such as at least 25 wt %, such as at least 30 wt %.

The salt may for example be a sodium salt or a calcium salt.

In one embodiment, the salt is selected from the group consisting of sodium chloride, sodium citrate, sodium dihydrogen phosphate and sodium dihydrogen phosphate.

In one embodiment, the concentration of salt in the BC suspension is at least 1 wt %, such as at least 5 wt %, such as at least 10 wt %, such as at least 15 wt %, such as at least 20 wt %.

Whether or not the BC suspension is stable in the above-mentioned conditions (i.e. a.-f.) can for example be tested using methods known in the art, for example using the methods described herein above in the section “Bacterial cellulose suspension produced from bacterial cellulose powder”.

In other words, if the BC suspension is stable in the above-mentioned conditions, it does not coagulate, flocculate and/or exhibit water-cellulose phase separation in said conditions.

Whether or not the BC suspension is stable in the above-mentioned conditions may also be tested by checking whether or not the viscosity of the BC suspension is maintained in the above-mentioned conditions, as compared to a BC suspension with the same properties but which has not been exposed to said above-mentioned conditions. If the viscosity is maintained, such as for example if at least 50%, such as at least 60%, such as at least 70%, such as at least 80, such as at least 90%, such as 100% of the viscosity is maintained, the BC suspension may be considered stable.

Viscosity can be measured using methods well known in the art, such as for example the methods described in Example 3.

The BC powder may be resuspended in an aqueous solution in any amount suitable to obtain a specific suitable concentration (e.g. wt %) of BC powder in said BC suspension.

In one embodiment, the BC suspension comprises at least 0.02 wt %, such as at least 0.04 wt %, such as at least 0.05 wt %, such as at least 0.1 wt %, such as at least 0.5 wt % BC powder.

In one embodiment, the BC suspension comprises between 0.02 and 3 wt % BC powder, such as between 0.02 and 2.4 wt %, such as between 0.02 and 2 wt %, such as between 0.03 and 2.8 wt %, such as between 0.03 and 2.4 wt %, such as between 0.03 and 2 wt %, such as between 0.04 and 2.8 wt %, such as between 0.04 and 2.4 wt %, such as between 0.04 and 2 wt %, such as between 0.05 and 1.5 wt %, such as between 0.04 and 1.2 wt % BC powder.

In one embodiment, the BC suspension has a viscosity of at least 1000 mPas, such as at least 1500 mPas, such as at least 2000 mPas, such as at least 2500 mPas at a concentration of 0.5 wt % BC powder, wherein the viscosity is measured at a shear rate of 1 s⁻¹ at 25° C.

In one embodiment, the BC suspension has a viscosity of at least 6000 mPas, such as at least 8000 mPas, such as at least 10,000 mPas at a concentration of 1.0 wt % BC powder, wherein the viscosity is measured at a shear rate of 1 s⁻¹ at 25° C.

In one embodiment, the BC suspension has a viscosity of at least 2000 mPas at a concentration of 0.5 wt % BC powder, wherein the viscosity is measured at a shear rate of 1 s⁻¹ at 25° C., and wherein said viscosity is maintained:

a. at a pH of between 2 and 12, such as between 3 and 11;

b. in the presence of surfactant, such as in the presence of at least 10 wt % surfactant;

c. in the presence of salt, such as such as in the presence of at least 10 wt % salt;

and/or
d. for at least 6 months of storage, such as for at least 9 months of storage, such as for at least 1 year of storage.

Preferably, the BC suspension prepared from the BC powder disclosed herein has a high water holding capacity, i.e. a water holding capacity of at least 80 g water per gram BC.

In one embodiment, the BC suspension has a water holding capacity of at least 80 g water/g BC (g/g), such as at least 90 g/g, such as at least 100 g/g, such as at least 110 g/g, such as at least 120 g/g, such as at least 130 g/g, such as at least 140 g/g, such as at least 150 g/g, such as at least 160 g/g, such as at least 170 g/g, such as at least 180 g/g, such as at least 190 g/g, such as at least 200 g/g.

In one embodiment, the BC suspension has a water holding capacity of between 80 and 200 g water/g BC (g/g), such as between 90 and 200 g/g such as between 90 and 180 g/g, such as between 100 and 150 g/g.

The water holding capacity of a BC suspension may be determined using the methods well known in the art. In particular, the water holding capacity of a BC suspension may be calculated based on how much water the cellulose pellet absorbs. For example, the water holding capacity may be determined by centrifuging the BC suspension at 4500×G for 20 minutes; removing the supernatant and weighing the pellet; and calculating the water holding capacity according to the formula below, where W is weight:

$$( {W}_{total} - {W}_{supematant} - {W}_{drypellett} ) / {W}_{drypellett}$$

Method for producing bacterial cellulose suspension from bacterial cellulose powder The present invention further relates to methods for producing BC suspensions from a BC powder, in particular from the BC powder described in the section “Bacterial cellulose powder”.

Hence, in one embodiment, the present invention relates to a method of producing a BC suspension, said method comprising mixing the BC powder as disclosed herein in the section “Bacterial cellulose powder” with an aqueous solution, thereby obtaining a BC suspension.

The BC suspension produced by said method does in particular possess one or more properties and characteristics that renders a stable BC suspension, as described above in the section “Bacterial cellulose suspension produced from bacterial cellulose powder”.

Thus, in a particular embodiment, the BC suspension is as disclosed in the section “Bacterial cellulose suspension produced from bacterial cellulose powder”.

In one embodiment, the method comprises or consists of the steps of
i. performing the method as described in the section “Method for producing bacterial cellulose suspension”, thereby obtaining the BC powder; and
ii. mixing the BC powder with an aqueous solution, thereby obtaining a BC suspension.

Uses of bacterial cellulose powder and bacterial cellulose suspension produced from bacterial cellulose powder

The re-dispersibility and several superior properties of the BC powder described herein as well as the stability and several superior properties of the BC suspension produced from the BC powder described herein opens for numerous applications.

The below-listed applications apply to both the BC powder and the BC suspension prepared from the BC powder as described herein.

Skin care. Due to being highly stable, the BC suspension does not phase separate or clump on the skin compared as frequently occurs with other MFC products and non-stabilized BC suspensions. Furthermore, due to the absence of stabilizing polymers the BC suspension has desirable sensorial properties i.e. non-sticky, non-greasy, non-stringy.

Thickening. Due to being highly dispersed and of high cellulose purity, the BC suspension is an effective thickener of aqueous liquids compared to MFC products and non-stabilized BC suspensions.

Solids-stabilization. Due to being highly stable and of high cellulose purity, the BC suspension is effective in stabilization of solids, in particular as compared to other MFC products and unstable BC suspensions.

Anti-dripping. Due to high water-holding capacity combined with its shear thinning properties, the BC suspension is effectively anti-dripping. This has implications especially for its use as rheology modifier in formulations which are pumped or sprayed onto a surface.

Film forming. Due to the film forming properties of the BC suspension, it has shown to be effective as an anti-wrinkle ingredient, and potentially also as a mattifying agent e.g., for hiding skin flaws or for cosmetic effects. For the formation of a homogenous film to occur (dry or wet) a stable BC suspension is required. The absence of other polymers which may not be film forming as well as minimal interference of polymer surface charges improves the film forming effect.

SPF booster. Due to being highly crystalline, of high purity and having the ability to stabilize water phase or particles in the formula, the BC suspension has shown to be an effective SPF (Sun Protection Factor) booster ingredient in sunscreen formulations. The crystalline regions of BC aids in blocking/scattering of UV light, and the dispersed fiber network aids in stabilizing particles such as ZnO and TiO2 which further enhances the intrinsic SPF of a sunscreen formulation.

### BC Powder

In one aspect, the present invention relates to use of the BC powder as disclosed herein as a thickener, stabilizer, emulsifier, co-emulsifier and/or rheology modifier.

In one aspect, the present invention relates to use of the BC powder as disclosed herein as a film-forming agent, sensorial enhancer, SPF-boosting agent and/or anti-wrinkle agent.

In one aspect, the present invention relates to use of the BC powder as disclosed herein as a reinforcer material.

The present invention further relates to use of the BC powder as disclosed herein in a method for preparing a composition as defined in the section “Compositions comprising bacterial cellulose powder bacterial cellulose suspension produced from bacterial cellulose powder”, or a product as defined in the section “Product comprising bacterial cellulose powder bacterial cellulose suspension produced from bacterial cellulose powder”.

BC Suspension Prepared from BC Powder

Thus, in one aspect, the present invention relates to use of the BC suspension produced from the BC powder as disclosed herein as a thickener, stabilizer, emulsifier, co-emulsifier and/or rheology modifier.

In one aspect, the present invention relates to use of the BC suspension produced from the BC powder as disclosed herein as a film-forming agent, sensorial enhancer, SPF-boosting agent and/or anti-wrinkle agent.

In one aspect, the present invention relates to use of the BC suspension produced from the BC powder as disclosed herein as a reinforcer material.

The present invention further relates to use of the BC suspension produced from the BC powder as disclosed herein in a method for preparing a composition as defined in the section “Compositions comprising bacterial cellulose powder bacterial cellulose suspension produced from bacterial cellulose powder”, or a product as defined in the section “Product comprising bacterial cellulose powder bacterial cellulose suspension produced from bacterial cellulose powder”.

Compositions Comprising Bacterial Cellulose Powder or Bacterial Cellulose Suspension Produced from Bacterial Cellulose Powder

The BC powder of the present invention and the BC suspension prepared from the BC powder of the present invention are particularly suitable for use in compositions, for example compositions with applications as described in the section “Use of bacterial cellulose powder and bacterial cellulose suspension produced from bacterial cellulose powder”. For example, such BC powders and BC suspensions as disclosed herein are suitable for personal care products, personal care, cosmetics, pharmaceutical products, biomedical products or food products.

The BC powder or BC suspension obtained from said powder may for example be included in a composition as a thickener, stabilizer, emulsifier, co-emulsifier, rheology modifier, film-forming agent, sensorial enhancer, SPF-boosting agent, anti-wrinkle agent and/or a reinforcer material.

Hence, provided herein is a composition comprising the BC powder as described in the section “Bacterial cellulose powder” or the BC suspension as described in the section “Bacterial cellulose suspension produced from bacterial cellulose powder”.

In one aspect, the present invention provides a composition comprising:
a. a resuspended BC powder, wherein the BC powder is as described in the section “Bacterial cellulose powder”, or
b. a BC suspension as described in the section “Bacterial cellulose suspension produced from bacterial cellulose powder”.

The BC powder or BC suspension may be added to any type of composition in any amount suitable for the specific composition.

In one embodiment, the composition is a dry composition, such as for example a powder. In one embodiment, the composition is a liquid composition, such as for example a suspension, a cream or a lotion.

In one embodiment, the concentration of BC powder in said composition is at least 0.01 wt %, such as at least 0.05 wt %, such as at least 0.1 wt %, such as at least 0.5 wt %, such as at least 1 wt %, such as at least 1.5 wt %, such as at least 2 wt %, such as at least 3 wt %, such as at least 4 wt %, such as at least 5 wt %, such as at least 10 wt %, such as at least 20 wt %, such as at least 50 wt %.

In one embodiment, the concentration of BC powder in said composition is between 0.01 wt % and 50 wt %, such as between 0.5 wt % and 25 wt %, such as between 0.5 wt % and 10 wt %, such as between 0.01 and 3 wt %, such as between 0.02 and 3 wt %, such as between 0.03 and 2.5 wt %, such as between 0.04 and 2 wt %.

The composition may comprise any additional ingredient suitable for the envisaged use, as known in the art. In one embodiment, the composition further comprises one or more ingredients selected from the group consisting of proteins, vitamins, peptides, beads, salts, oils, particles, humectants, pH adjusters, pH buffers, preservatives, silicones, antioxidants, disinfectants, antimicrobials, emollients, chelating agents, colorants, fragrances, solvents and surfactants.

Thus, in one embodiment, the present invention provides a composition comprising:
a. a resuspended BC powder, wherein the BC powder is as described in the section “Bacterial cellulose powder”, or
b. a BC suspension as described in the section “Bacterial cellulose suspension produced from bacterial cellulose powder”
   and one or more ingredients selected from the group consisting of proteins, vitamins, peptides, beads, salts, oils, particles, humectants, pH adjusters, pH buffers, preservatives, silicones, antioxidants, disinfectants, antimicrobials, emollients, chelating agents, colorants, fragrances, solvents and surfactants.

The surfactant may for example be a cationic surfactant, a zwitterionic surfactant, an anionic surfactant, an amphoteric surfactant and/or a nonionic surfactant.

In one embodiment, the surfactant is selected from the group consisting of cocoamidpropyl betaine (CAPB), sodium dodecyl sulfate (SDS), sodium laureth sulfate (SLS), lauryl glucoside and surfactants comprising quaternary ammonium cation (QUAT), decyl glucose, polyquaternium-7 (QUAT 7), behentrimonium chloride, glyceryl stearate, sodium cetearyl sulphate, glyceryl caprylate, sorbitan laurate/C18-C20 and glycol isostearate.

In one embodiment, the concentration of surfactant in the composition is at least 5 wt %, such as at least 10 wt %, such as at least 15 wt %, such as at least 20 wt %, such as at least 25 wt %, such as at least 30 wt %.

The salt may for example be a sodium salt or a calcium salt.

In one embodiment, the salt is selected from the group consisting of sodium chloride, sodium citrate, sodium dihydrogen phosphate and sodium dihydrogen phosphate.

In one embodiment, the concentration of salt in the composition is at least 1 wt %, such as at least 5 wt %, such as at least 10 wt %, such as at least 15 wt %, such as at least 20 wt %.

The vitamin may for example be vitamin A and/or vitamin E.

The particle or bead may for example be selected from the group consisting of pearlescents; decorative beads; fragrance beads; gelatine beads; jojoba beads; polyethylene beads; charcoal particles; apricot kernel particles; colorant particles; pigments; air bubbles; insoluble enzymes; encapsulated actives such as moisturizers or exfoliating agents such as alpha-hydroxy acids and glycolic acid; inorganic minerals; kaolin; zeolites; TiO2 and ZnO2.

In one embodiment, the particle or bead may comprise an active ingredient such as:
i. a vitamin, such as vitamin A or vitamin E;
ii. retinol, or
iii. an active ingredient which is not soluble in water but is suspendable in water or oil.

The oil may for example be in the form of oil droplets; oil suspensions; or oil emulsions. In one embodiment, the oil is one or more oil selected form the group consisting of paraffin and jojoba oil.

In one embodiment, the composition comprising the resuspended BC powder or the BC suspension is stable. Hence, preferably, the composition comprising the BC powder or the BC suspension does not flocculate, coagulate and/or does not exhibit water-cellulose separation.

In one embodiment, the composition:
a. is stable;
b. does not clump;
c. does not flocculate;
d. does not exhibit water-cellulose separation;
e. is capable of stabilizing particles; and/or
f. maintains its viscosity;
   in the following conditions:
for at least 6 months of storage, such as for at least 9 months of storage, such as for at least 1 year of storage;
at a pH of between 2 and 12, such as at a pH of between 3 and 11; and/or
at high temperatures.

Whether or not the composition is stable and/or does not coagulate, does not flocculate and/or does not water-cellulose phase separate can be tested as described above in the section “Bacterial cellulose suspension produced from bacterial cellulose powder”.

In one embodiment, the composition does not comprise any additional thickener, stabilizer and/or rheology modifier. In one embodiment, the composition does not comprise any additional polymeric thickener, such as for example a natural polymer, such as for example carboxymethyl cellulose and/or natural gums.

In one embodiment, the composition is aqueous.

In one embodiment, the composition comprises a polyol, such as glycerol or propane diol.

In one embodiment, the present invention provides a composition comprising:
a. a resuspended BC powder, wherein the BC powder is as described in the section “Bacterial cellulose powder, or a BC suspension as described in the section “Bacterial cellulose suspension produced from bacterial cellulose powder”; and
b. water; and
c. a compound selected from the group consisting of surfactant, emulsifier, salt, buffer, particles, thickener, stabilizer and preservative.

In one embodiment, the present invention provides a composition comprising:
a. a resuspended BC powder, wherein the BC powder is as described in the section “Bacterial cellulose powder, or a BC suspension as described in the section “Bacterial cellulose suspension produced from bacterial cellulose powder”; and
b. water; and
c. a compound selected from the group consisting of surfactant, emulsifier and glycerine.

The surfactant, the salt and/or the oil may be as defined herein in the section

“Compositions comprising bacterial cellulose powder or bacterial cellulose suspension prepared from bacterial cellulose powder”.

In one embodiment, the present invention provides a composition comprising:
a. a resuspended BC powder, wherein the BC powder is as described in the section “Bacterial cellulose powder, or a BC suspension as described in the section “Bacterial cellulose suspension produced from bacterial cellulose powder”; and
b. water; and
c. a quaternary ammonium compound (QUAT), such as a polyquat.

In one embodiment, the concentration of BC powder in said composition is between 0.05 and 3 wt %, for example between 0.1 and 1 wt %.

In one embodiment, the concentration of QUAT in said composition is between 2 and 20 wt %, such as between 5 and 15 wt %.

In one embodiment, the present invention provides a composition comprising:
a. 0.01 to 3 wt % resuspended BC powder, wherein the BC powder is as described in the section “Bacterial cellulose powder”;
b. 60 to 90 wt % water;
C. 1 to 10 wt % glycerine;
d. 5 to 20 wt % surfactant;
e. optionally, 0.1 to 1% preservative;
f. optionally, comprises buffer.

Products comprising bacterial cellulose powder or bacterial cellulose suspension produced from bacterial cellulose powder

The BC powder of the present invention as well as the BC suspension prepared from the BC powder of the present invention is further highly useful in certain products, for example, but not limited to, products related to the uses described in the section “Uses of bacterial cellulose powder and bacterial cellulose suspension produced from bacterial cellulose powder”.

Hence, provided herein is a product comprising the BC powder as described herein in the section “Bacterial cellulose powder”.

Further provided herein is a product comprising the BC suspension as described herein in the section “Bacterial cellulose suspension produced from bacterial cellulose powder”.

Further provided herein is a product comprising the composition as described herein in the section “Compositions comprising bacterial cellulose powder or bacterial cellulose suspension produced from bacterial cellulose powder”.

In one embodiment, the product is selected from the group consisting of personal care products, cosmetic products, pharmaceutical products, biomedical products and food products.

In one embodiment, the cosmetic product is selected from the group consisting of lipstick, mascara, foundation, highlighter, primer, concealer and nail polish.

In one embodiment, the personal care product is selected from the group consisting of cream, lotion, gel, oil, foam, balm, pomade, moisturizer, serum, soap, detergent and scrub, for example wherein the product is facial cleanser, toothpaste, sunscreen, sunblock, shampoo, hair conditioner, hair oil, body lotion, body wash, shower gel, lip balm, shaving cream, shaving gel, deodorant, hand soap, eye cream, eye serum, face cream, anti-wrinkle cream and hand cream.

In one embodiment, the pharmaceutical product is selected from the group consisting of a cream, lotion, gel, oil, foam, balm, pomade, moisturizer, serum, soap, detergent and scrub.

### Items

1. A bacterial cellulose (BC) powder comprising:
   a. BC; and
   b. homogalacturonan (HG);
wherein the ratio of HG: BC is at least 0.02:1 (wt:wt).
2. The BC powder according to item 1, wherein the HG: BC ratio is at least 0.05:1, such as at least 0.1:1, such as at least 0:15:1, such as at least 0.2:1, such as at least 0.5:1, such as at least 1:1.
3. The BC powder according to any one of the preceding items, wherein the HG: BC ratio is at most 50:1, such as at most 20:1, such as at most 10:1, such as at most 5:1, such as at most 2:1.
4. The BC powder according to any one of the preceding items, wherein the HG: BC ratio is between 0:02:1 and 50:1, such as between 0.05:1 and 20:1, such as between 0.05:1 and 10:1, such as between 0.1:1 and 10:1, such as between 0.1:1 and 5:1, such as between 0.1:1 and 1:1, such as between 0.1:1 and 0.5:1.
5. The BC powder according to any one of the preceding items, wherein the BC has a crystallinity of at least 30%, such as at least 40%, such as at least 50%, such as at least 60%, such as at least 70%, such as at least 80%, wherein the crystallinity is determined with Fourier Transform Infrared spectroscopy (FT-IR) by calculating the peak ratio at 1430 and 898 cm-1.
6. The BC powder according to any one of the preceding items, wherein the BC has a crystallinity of between 30% and 100%, such as between 50% and 100%, such as between 40% and 90%, such as between 60% and 90%, such as between, 70% and 90%, such as between 70% and 85%, wherein the crystallinity is determined with FT-IR by calculating the peak ratio at 1430 and 898 cm⁻¹.

7. The BC powder according to any one of the preceding items, wherein the BC powder comprises powder particles with an average powder particle size of between 0.1 and 500 μm, such as between 1 and 50 μm, such as between 10 and 40 μm, such as between 15 and 30 μm, such as between 19 and 24 μm, wherein the average powder particle size is calculated based on the powder particle volume distribution.

8. The BC powder according to any one of the preceding items, wherein the BC powder comprises powder particles with an average powder particle size of between 0.01 and 200 μm, such as between 0.1 and 20 μm, such as between 1 and 15 μm, such as between 1 and 10 μm, such as between 2 and 9 μm, such as between 4 and 8 μm, wherein the average powder particle size is calculated based on the powder particle number distribution.

9. The BC powder according to any one of the preceding items, wherein the BC powder comprises powder particles with an average convexity of at least 0.8, such as at least 0.85, such as at least 0.9, such as at least 0.92, such as at least 0.94, such as at least 0.96, such as at least 0.97, wherein the average convexity is calculated based on the powder particle number distribution or based on the powder particle volume distribution.

10. The BC powder according to any one of the preceding items, wherein the BC powder comprises powder particles with a smooth surface.

11. The BC powder according to any one of the preceding items, wherein the BC powder comprises powder particles with an average elongation of between 0.4 and 0.7, such as between 0.45 and 0.65, wherein the average elongation is calculated based on the powder particle volume distribution.

12. The BC powder according to any one of the preceding items, wherein the BC powder comprises powder particles with an average elongation of between 0.1 and 0.4, such as between 0.2 and 0.3, wherein the average elongation is calculated based on the powder particle number distribution.

13. The BC powder according to any one of the preceding items, wherein the BC powder comprises elliptical-shapes powder particles.

14. The BC powder according to any one of the preceding items, wherein the BC powder comprises powder particles with an average circularity of at least 0.8, such as at least 0.85, such as at least 0.9, wherein the average circularity is calculated based on the powder particle volume distribution.

15. The BC powder according to any one of the preceding items, wherein the BC powder comprises powder particles with an average circularity of at least 0.7, such as at least 0.75, such as at least 0.8, wherein the average circularity is calculated based on the powder particle number distribution.

16. The BC powder according to any one of the preceding items, wherein the BC powder is re-dispersible in aqueous media by zero shear or low shear mixing.

17. The BC powder according to any one of the preceding items, wherein the BC powder is re-dispersible in aqueous media by mixing with a deflocculator, such as a deflocculator mixer, for example a deflocculator turbine.

18. The BC powder according to any one of the preceding items, wherein the BC powder is re-dispersible in aqueous media by mixing at between 300 rpm and 20,000 rpm, such as between 500 rpm and 5000 rpm, such as between 600 rpm and 10,000 rpm, optionally by mixing for between 30 seconds and 25 minutes, such as by mixing for between 1 and 20 minutes.

19. The BC powder according to any one of the preceding items, wherein the rhamnogalacturonan (RG): BC ratio is at most 1:1, such as at most 0.5:1, such as at most 0.1:1, such as at most 0.01:1, such as at most 0.005:1.

20. The BC powder according to any one of the preceding items, wherein the HG upon breakdown comprises at least 65% galacturonic acid (GA), such as at least 70%, such as at least 70%, such as at least 75%, such as at least 80%, such as at least 85%, such as at least 90%, such as at least 95% GA.

21. The BC powder according to item 19, wherein the RG upon breakdown comprises less than 65% GA, such as less than 60%, such as less than 55%, such as less than 50%, such as less than 45%, such as less than 40%, such as less than 35%, such as less than 30% GA.

22. The BC powder according to any one of the preceding items, wherein the HG is comprised in pectin.

23. The BC powder according to item 22, wherein the pectin comprises at least 70% HG, such as at least such as at least 75%, such as at least 80%, such as at least 85%, such as at least 90%, such as at least 95% HG.

24. The BC powder according to any one of items 22 to 23, wherein the pectin comprises at most at most 35% RG, such as at most 30% RG, such as at most 25% RG, such as at most 20% RG, such as at most 15%, such as at most 10% RG.

25. The BC powder according to any one of items 22 to 24, wherein the pectin upon breakdown comprises at least 65% GA, such as at least 70%, such as at least 75%, such as at least 80%, such as at least 85%, such as at least 90%, such as at least 95% GA.

26. The BC powder according to any one of the preceding items, wherein the BC powder has a recovery of viscosity of at least 40% upon redispersion, such as at least 50%, such as at least 60%, such as at least 70%, such as at least 80%, such as at least 90%, such as a recovery of viscosity of 100% upon redispersion, wherein the recovery of viscosity upon redispersion is relative to the viscosity of a corresponding never-dried BC suspension having the same concentration of BC, and wherein the recovery of viscosity is calculated using the formula:

$$Viscosity recovery ( \% ) = 100 * \frac{Viscosity ( at 1 s - 1 ) of redispersed BC powder}{Viscosity ( at 1 s - 1 ) of never dried BC suspension}$$

27. The BC powder according to any one of the preceding items, wherein the BC powder has a recovery of water holding capacity of at least 50% upon redispersion, such as at least 60%, such as at least 70%, such as at least 80%, such as at least 90%, such as a recovery of water holding capacity of 100% upon redispersion, wherein the recovery of water holding capacity is relative to the viscosity of a corresponding never-dried BC suspension, and wherein the recovery of viscosity is calculated using the formula:

$$Water holding capacity revovery ( \% ) = 100 * \frac{Water holding capacity of redispersed BC powder}{Water holding capacity of never dried BC suspension}$$

28. A method for producing a BC powder, said method comprising providing a BC suspension and:
b. processing said BC suspension;
c. recovering said BC suspension;
d. mixing said BC suspension with HG, thereby obtaining a BC-HG suspension;
e. drying the BC-HG suspension;
   thereby obtaining a BC powder
   wherein the ratio of HG: BC of said BC powder is at least 0.02:1 (wt:wt).

29. A method for producing a BC powder, said method comprising the steps of:
a. incubating a cellulose-producing bacteria in a culture medium, thereby obtaining a fermentation broth comprising a BC suspension;
b. processing said BC suspension;
c. recovering said BC suspension;
d. mixing said BC suspension with HG, thereby obtaining a BC-HG suspension;
e. drying the BC-HG suspension;
   thereby obtaining a BC powder,
   wherein the ratio of HG: BC of said BC powder is at least 0.02:1 (wt:wt).

30. The method according to any one of items 28 to 29, wherein the BC powder is as defined in any one of items 1 to 27.

31. The method according to any one of items 29 to 30, wherein the cellulose-producing bacteria is of a genus selected from the group consisting of Acetobacter, Gluconacetobacter, Komagataeibacter and Gluconobacter.

32. The method according to any one of items 29 to 31, wherein the cellulose-producing bacteria is of a species selected from the group consisting of Acetobacter xylinus, Acetobacter xylinum, Acetobacter hansenii, Acetobacter pasteurianus, Gluconacetobacter xylinus, Gluconobacter oxydans, Komagataeibacter rhaeticus and Komagataeibacter xylinus, preferably wherein the cellulose-producing bacteria is Komagataeibacter xylinus.

33. The method according to any one of items 29 to 32, wherein the incubation of step a. takes place in an agitated reactor, such as in a stirred tank reactor.

34. The method according to any one of items 29 to 33, wherein the incubation of step a. is between one and six days, such as between two and five days.

35. The method according to any one of items 28 to 34, wherein step b. comprises or consists of:
i. washing the recovered BC suspension with water;
ii. treating the BC suspension with an alkali, preferably with sodium hydroxide;
iii. neutralizing the BC suspension with acid; and/or
iv. separating the BC from the fermentation broth, optionally by filtration.

36. The method according to any one of items 28 to 35, wherein step d. comprises homogenization of the BC-HG suspension.

37. The method according to any one of items 28 to 36, wherein step e. comprises or consists of spray drying or oven drying, preferably wherein step e. comprises spray drying.

38. The method according to item 35, wherein step ii. is performed using sodium hydroxide with a concentration of between 0.2 to 0.8 M, preferably at a temperature of between 5° and 80° C.

39. A BC suspension comprising the BC powder according to any one of items 1 to 27 resuspended in an aqueous solution.

40. The BC suspension according to item 39, wherein said BC suspension has a viscosity of at least 1000 mPas, such as at least 1500 mPas, such as at least 2000 mPas, such as at least 2500 mPas at a concentration of 0.5 wt % BC powder, wherein the viscosity is measured at a shear rate of 1 s⁻¹ at 25° C.

41. The BC suspension according to any one of items 26 to 40, wherein said BC suspension has a viscosity of at least 6000 mPas, such as at least 8000 mPas, such as at least 10,000 mPas at a concentration of 1.0 wt % BC powder, wherein the viscosity is measured at a shear rate of 1 s⁻¹ at 25° C.

42. The BC suspension according to any one of items 26 to 41, wherein said BC suspension is capable of stabilizing particles.

43. The BC suspension according to item 42, wherein said particles have a size of between 0.1 mm and 5 mm in diameter, such as between 0.25 mm and 0.65 m, such as between 1 mm and 2 mm, such as between 1 mm and 1.5 mm, such as between 0.25 mm and 2 mm, such as between 0.1 mm and 4 mm, such as between 0.1 mm and 3 mm.

44. The BC suspension according to any one of items 42 to 43, wherein said particles are selected from the group consisting of pearlescents; decorative beads; fragrance beads; gelatine beads; jojoba beads; polyethylene beads; charcoal particles; apricot kernel particles; colorant particles; pigments; air bubbles; insoluble enzymes; encapsulated actives such as moisturizers or exfoliating agents such as alpha-hydroxy acids and glycolic acid; inorganic minerals; kaolin; zeolites; TiO2; ZnO2; vitamins such as vitamin A or vitamin E; oil droplets; oil suspensions; and oil emulsions, optionally wherein said particles comprise an active ingredient such as:
i. a vitamin, preferably vitamin A or vitamin E;
ii. retinol, or
iii. an active ingredient which is not soluble in water but is suspendable in water or oil.

45. The BC suspension according to any one of items 26 to 44, wherein said BC suspension:
a. is stable;
b. does not clump;
c. does not flocculate;
d. does not exhibit water-cellulose separation;
e. is capable of stabilizing particles; and/or
f. maintains its viscosity;
   for at least 6 months of storage, such as for at least 9 months of storage, such as for at least 1 year of storage.

46. The BC suspension according to any one of items 26 to 45, wherein said BC suspension:
a. is stable;
b. does not clump;
c. does not flocculate;
d. does not exhibit water-cellulose separation;
e. is capable of stabilizing particles; and/or
f. maintains its viscosity;
   at a pH of between 2 and 12, such as at a pH of between 3 and 11.

47. The BC suspension according to any one of items 26 to 46, wherein said BC suspension:
a. is stable;
b. does not clump;
c. does not flocculate;
d. does not exhibit water-cellulose separation;
e. is capable of stabilizing particles; and/or
f. maintains its viscosity;
   in the presence of a surfactant,
   optionally wherein the surfactant is a cationic surfactant, a zwitterionic surfactant, an anionic surfactant, an amphoteric surfactant and/or a nonionic surfactant,
   further optionally wherein the surfactant is selected from the group consisting of cocoamidpropyl betaine (CAPB), sodium dodecyl sulfate (SDS), sodium laureth sulfate (SLS), lauryl glucoside and surfactants comprising quaternary ammonium cation (QUAT), decyl glucose, polyquaternium-7 (QUAT 7), behentrimonium chloride, glyceryl stearate, sodium cetearyl sulphate, glyceryl caprylate, sorbitan laurate/C18-C20 and glycol isostearate,
   further optionally wherein the concentration of surfactant in the BC suspension is at least 5 wt %, such as at least 10 wt %, such as at least 15 wt %.

48. The BC suspension according to any one of items 26 to 47, wherein said BC suspension:
a. is stable;
b. does not clump;
c. does not flocculate;
d. does not exhibit water-cellulose separation;
e. is capable of stabilizing particles; and/or
f. maintains its viscosity;
   in the presence of a salt,
   optionally wherein the salt is a sodium salt or a calcium salt,
   preferably wherein the salt is selected from the group consisting of sodium chloride, sodium citrate, sodium dihydrogen phosphate and sodium dihydrogen phosphate,
   optionally wherein the concentration of salt in the BC suspension is at least 5 wt %, such as at least 10 wt %, such as at least 15 wt %.

49. The BC suspension according to any one of items 26 to 48, wherein said BC suspension:
a. is stable;
b. does not clump;
c. does not flocculate;
d. does not exhibit water-cellulose separation;
e. is capable of stabilizing particles; and/or
f. maintains its viscosity;
   in the presence of one or more compounds selected from the group consisting of surfactant, salt, buffer, particles, emulsifier, thickener, stabilizer and preservative.

50. The BC suspension according to any one of items 26 to 49, wherein the aqueous solution is water.

51. The BC suspension according to any one of items 26 to 50, wherein said BC suspension comprises at least 0.02 wt %, such as at least 0.04 wt %, such as at least 0.05 wt %, such as at least 0.1 wt %, such as at least 0.5 wt % BC powder.

52. The BC suspension according to any one of items 26 to 50, wherein said BC suspension comprises between 0.02 and 3 wt % BC powder, such as between 0.02 and 2.4 wt %, such as between 0.02 and 2 wt %, such as between 0.03 and 2.8 wt %, such as between 0.03 and 2.4 wt %, such as between 0.03 and 2 wt %, such as between 0.04 and 2.8 wt %, such as between 0.04 and 2.4 wt %, such as between 0.04 and 2 wt %, such as between 0.05 and 1.5 wt %, such as between 0.04 and 1.2 wt % BC powder.

53. The BC suspension according to any one of items 26 to 52, wherein said BC suspension has a water holding capacity of at least 100 g water per g BC (g/g), such as at least 120 g/g, such as at least 130 g/g, such as at least 140 g/g, such as at least 150 g/g.

54. The BC suspension according to any one of items 26 to 53, wherein said BC suspension has a viscosity of at least 2000 mPas at a concentration of 0.5 wt % BC powder,
wherein the viscosity is measured at a shear rate of 1 s⁻¹ at 25° C., and wherein said viscosity is maintained:
   a. at a pH of between 2 and 12, such as between 3 and 11;
   b. in the presence of surfactant, such as in the presence of at least 10 wt % surfactant;
   c. in the presence of salt, such as such as in the presence of at least 10 wt % salt;
   d. for at least 6 months of storage, such as for at least 9 months of storage, such as for at least 1 year of storage.

55. A method for preparing a BC suspension, said method comprising mixing the BC powder of any one of items 1 to 27 with an aqueous solution, thereby obtaining a BC suspension.

56. The method according to item 55, wherein the BC suspension is as defined in any one of items 39 to 53.

57. The method according to any one of items 55 to 56, the method comprising or consisting of the steps of:
i. performing the method according to any one of items 28 to 38, thereby obtaining the BC powder; and
ii. mixing the BC powder with an aqueous solution, thereby obtaining the BC suspension.

58. Use of the BC powder according to any one of items 1 to 27 or the BC suspension according to any one of items 39 to 53 as a thickener, stabilizer, emulsifier, co-emulsifier and/or rheology modifier.

59. Use of the BC powder according to any one of items 1 to 27 or the BC suspension according to any one of items 39 to 53 as a film-forming agent, sensorial enhancer, SPF-boosting agent and/or anti-wrinkle agent.

60. Use of the BC powder according to any one of items 1 to 27 or the BC suspension according to any one of items 39 to 53 as a reinforcer material.

61. A composition comprising:
a. a resuspended BC powder, wherein the BC powder is the BC powder according to any one of items 1 to 27; or
b. the BC suspension according to any one of items 39 to 53.

62. The composition according to item 61, wherein the concentration of BC powder in said composition is between 0.01 and 3 wt %, such as between 0.02 and 3 wt %, such as between 0.03 and 2.5 wt %, such as between 0.04 and 2 wt %.

63. The composition according to any one of items 61 to 62, wherein said composition further comprises one or more ingredients selected from the group consisting of proteins, vitamins, peptides, beads, salts, oils, particles, humectants, pH adjusters, pH buffers, preservatives, silicones, antioxidants, disinfectants, antimicrobials, emollients, chelating agents, colorants, fragrances, solvents and surfactants.

64. The composition according to item 63, wherein:
a. the surfactant is one or more surfactant selected from the group consisting of cationic surfactant, zwitterionic surfactant, anionic surfactant, amphoteric surfactant and nonionic surfactant, optionally wherein the surfactant is selected from the group consisting of cocoamidpropyl betaine (CAPB), sodium dodecyl sulfate (SDS), sodium laureth sulfate (SLS), lauryl glucoside and surfactants comprising quaternary ammonium cation (QUAT), decyl glucose, polyquaternium-7 (QUAT 7), behentrimonium chloride, glyceryl stearate, sodium cetearyl sulphate, glyceryl caprylate, sorbitan laurate/C18-C20 and glycol isostearate;
b. the particle is one or more particle selected from the group consisting of pearlescents; decorative beads; fragrance beads; gelatine beads; jojoba beads; polyethylene beads; charcoal particles; apricot kernel particles; colorant particles; pigments; air bubbles; insoluble enzymes; encapsulated actives such as moisturizers or exfoliating agents such as alpha-hydroxy acids and glycolic acid; inorganic minerals; kaolin; zeolites; TiO2; ZnO2; vitamins such as vitamin A or vitamin E; oil droplets; oil suspensions; and oil emulsions; and/or
c. the oil is one or more oil selected form the group consisting of paraffin and jojoba oil.

65. The composition according to any one of items 61 to 64, wherein the composition:
a. is stable;
b. does not clump;
c. does not flocculate; and/or
d. does not exhibit water-cellulose separation,
   after at least 3 months of storage, such as after at least 4 months of storage, such as after at least 6 months of storage, such as after at least 1 year of storage.

66. The composition according to any one of items 61 to 65, wherein said composition does not comprise any additional thickener, stabilizer and/or rheology modifier, optionally wherein said composition does not comprise any additional polymeric thickener, such as for example a natural polymer, such as for example carboxymethyl cellulose and/or natural gums.

67. The composition according to any one of items 61 to 66, wherein the composition is aqueous.

68. The composition according to any one of items 61 to 66, wherein the composition comprises a polyol, such as glycerol or propane diol.

69. A composition comprising:
a. a resuspended BC powder, wherein the BC powder is the BC powder according to any one of items 1 to 27;
b. water; and
c. a compound selected from the group consisting of surfactant, emulsifier, salt, buffer, particles, thickener, stabilizer and preservative.

70. The composition according to item 69, wherein the surfactant, the salt and/or the oil is as defined in item 64.

71. A composition comprising:
a. a resuspended BC powder, wherein the BC powder is the BC powder according to any one of items 1 to 27;
b. water; and
c. a compound selected from surfactant, emulsifier and glycerine.

72. A composition comprising:
a. a resuspended BC powder, wherein the BC powder is the BC powder according to any one of items 1 to 27;
b. water; and
c. a quaternary ammonium compound (QUAT), such as a polyquat;
   optionally wherein the concentration of BC powder is between 0.05 and 3 wt %, for example between 0.1 and 1 wt %, further optionally wherein the concentration of QUAT is between 2 and 20 wt %, such as between 5 and 15 wt %.

73. A composition comprising:
a. 0.01 to 3 wt % of resuspended BC powder, wherein the BC powder is the BC powder according to any one of items 1 to 27;
b. 60 to 90 wt % water;
c. 1 to 10 wt % glycerine;
d. 5 to 20 wt % surfactant; and
e. 0.1 to 1% preservative;
   optionally wherein the composition further comprises buffer.

74. A product comprising the BC powder according to any one of items 1 to 27; the BC suspension according to any one of items 39 to 53; or the composition according to any one of items 61 to 73.

75. The product according to item 74, wherein said product is selected from the group consisting of personal care products, cosmetic products, pharmaceutical products, biomedical products and food products.

76. The product according to item 75, wherein the cosmetic product is selected from the group consisting of lipstick, mascara, foundation, highlighter, primer, concealer and nail polish.

77. The product according to item 75, wherein the personal care product is selected from the group consisting of cream, lotion, gel, oil, foam, balm, pomade, moisturizer, serum, soap, detergent and scrub, for example wherein the product is facial cleanser, toothpaste, sunscreen, sunblock, shampoo, hair conditioner, hair oil, body lotion, body wash, shower gel, lip balm, shaving cream, shaving gel, deodorant, hand soap, eye cream, eye serum, face cream, anti-wrinkle cream and hand cream.

### Certain Embodiments

A problem to be solved by the present invention relates to the provision of a novel improved re-dispersible (alternatively expressed re-suspensible) bacterial cellulose (BC) powder composition with reduced loss of pre-drying characteristics (e.g. improved recovery of viscosity, gel properties, and dispersibility upon resuspension with water).

As discussed above-a re-dispersible (alternatively expressed re-suspensible) BC powder composition with reduced loss of pre-drying characteristics (e.g. improved recovery of viscosity upon resuspension) may be of significant commercial interest-reasons for this relate e.g. to that it may be easier to transport a commercially relevant BC powder product as compared to a BC suspension product and integration of such product to end product formula such as in personal, medical and home care.

However, and as discussed in further detail herein (see e.g. Examples)—the present inventors anyway tested different commercially available pectin products and found surprisingly good results for a preferred amount of pectin homogalacturonan (HG) in combination with BC having a preferred crystallinity index (CI).

Below are briefly discussed some of the herein relevant technical results of working examples.

### Example 3

FIG. 3 discussed in this example—shows the viscosity profile of a preferred BC powder composition with BC:HG ratio of 1:0.1 measured as “Never dried” and “Redispersed”. There was very little difference—i.e. the BC:HG ratio of 1:0.1 gave a very good re-dispersible (alternatively expressed re-suspensible) BC powder composition with reduced loss of pre-drying characteristics (i.e. good recovery of viscosity upon resuspension). The results of the Example also showed that higher ratios/concentrations of HG did not significantly change the positive results—i.e. higher ratios/concentrations of HG may be used, even though maybe not preferred for a number of commercial relevant uses—where use of as small as possible amounts of HG may be preferred. The results also demonstrated that rhamnogalacturonan (RG) did not work as good as HG—i.e. one may say HG worked surprisingly good as compared to RG.

### Example 4

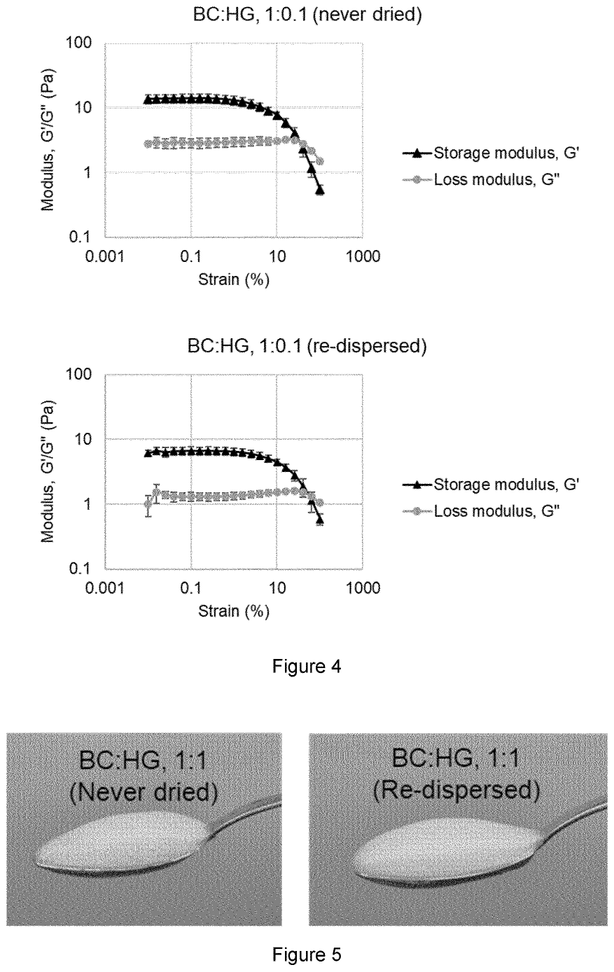

FIG. 4 discussed in this example—shows the storage and loss modulus profiles (elasticity/gel strength) of the same preferred BC powder composition of Example 3—also measured as “Never dried” and “Redispersed”. There was little difference—i.e. the BC:HG ratio of 1:0.1 gave a very good re-dispersible (alternatively expressed re-suspensible) BC powder composition with reduced loss of pre-drying characteristics (i.e. good recovery of elasticity/gel strength upon resuspension). Like Example 3—the results of this Example also showed that higher ratios/concentrations of HG did not significantly change the positive results. The results also demonstrated that rhamnogalacturonan (RG) did not work as good as HG—i.e. one may say HG worked surprisingly good as compared to RG.

### Example 5

Viscosity recovery and storage modulus recovery were repeated according to Example 3 and 4 above for a BC powder with a BC:HG ratio of 1:1 that comprises non-functionalized cellulose. The results were very positive—i.e. the HG seems to have even better effect on re-dispersibility of non-functionalized BC. The recovery of viscosity upon re-dispersion was 97% and recovery of storage modulus (elasticity/gel strength) upon re-dispersion was 100%.

Without being limited to theory—it is believed that the results of e.g. the working examples herein make it plausible that good BC powder re-dispersible results may be obtained at least over a BC:HG ratio range of the first aspect discussed below. It may be that a BC:HG ratio of 1:0.1 or a BC:HG ratio of 1:1 may be preferred for a particular BC powder of interest—but there is no reason to believe that commercial relevant acceptable good result should not be obtainable over a suitable broader range around these possible preferred ratios.

The BC powder compositions of the working Examples herein had a relatively high crystallinity index (CI)—which may be seen as an indication that the BC powder compositions contain intact natural cellulose fibers. Without being limited to theory-a BC powder composition with a relatively high crystallinity index (CI) may be seen as a powder composition with a higher risk of not being able to re-disperse (re-suspend) properly without losing its high CI related characteristics, which functionally may relate to e.g. loss of viscosity profile and loss of elasticity/gel strength. Accordingly, the very good positive re-dispersion (re-suspension) results illustrated in e.g. FIGS. 3 and 4 herein may objectively be seen as surprisingly good results.

Accordingly, a first aspect of the present invention relates to a bacterial cellulose (BC) powder composition comprising:
(i): a BC: homogalacturonan (HG) ratio from 1:0.02 to 1:10000 (w/w); and
(ii): wherein the BC has a crystallinity index (CI) of 0.05 to 8, determined with Fourier Transform Infrared spectroscopy (FT-IR) by calculating the peak ratio at 1430 and 898 cm⁻¹.

It is routine work for the skilled person to determine the amount of BC, HG and RG in a herein relevant (BC) powder composition of interest (see e.g. Barnes et al., 2021)—i.e. it is routine work to determine the BC:HG and BC: RG ratios for a relevant (BC) powder composition of interest.

Just as an example, a BC:HG ratio of 1:0.1 (w/w) may be obtained by adding 10g HG to 100g BC.

WO2022/200631A1 (Cellugy, DK) describes different BC production methods (e.g. specific culture conditions-such as use of different carbon sources in a static medium) to obtain BC with a crystallinity index of the first aspect—i.e. it is routine work for the skilled person to obtain BC with a crystallinity index of the first aspect. See e.g. also working Example 1 herein describing a preferred production method to obtain a preferred relatively high BC crystallinity index. As known in the art (see e.g. O'Connor et al., 1958) —to determine CI with FT-IR in a BC powder composition of the first aspect is routine work for the skilled person.

A second aspect of the present invention relates to a method for producing a BC powder composition of the first aspect and/or embodiments thereof comprising the steps of:
(a): Obtaining a broth from a fermenter comprising BC with a crystallinity index (CI) of the first aspect and/or embodiment thereof;
(b): Purifying the BC from the broth of step (a);
(c): Optionally, contacting the purified BC of step (b) with a functionalization agent to thereby functionalizing the BC;
(d): Optionally, comminution of the BC from step (b) and/or step (c) to get a BC particle size of interest;
(e): Adding HG to the BC of step (b), step (c), and/or step (d) in an amount that gives a BC:HG ratio from 1:0.02 to 1:10000 (w/w);
(f): Drying the BC with added HG of step (e) to get a BC powder composition of the first aspect and/or embodiment thereof.

Based on the technical teaching herein and the common general knowledge—the skilled person may routinely perform the individual steps of the method of the second aspect—i.e. may routinely perform the method of the second aspect as such.

As understood by the skilled person in the present context—by re-dispersing (re-suspending) a BC powder composition of the first aspect one may get a BC suspension—i.e. any commercial relevant known use of a BC suspension (see e.g. WO2022/200631A1 (Cellugy)) may also be a commercial relevant known use of a BC powder composition of the first aspect. Accordingly, a third aspect of the present invention relates to a method to obtain a product of interest, wherein the method comprises a step of using a BC powder composition of the first aspect and/or embodiments thereof to obtain the product of interest and other suitable steps to obtain the product of interest.

The third aspect may alternatively be expressed as use of a BC powder composition of the first aspect and/or embodiments thereof for making a product of interest. Based on the technical teaching herein and the common general knowledge—the skilled person may routinely perform the individual steps of the method of the third aspect—i.e. may routinely perform the method of the third aspect as such.

As understood by the skilled person in the present context—the use of the third aspect to make a product of interest may involve a step of re-suspending the BC powder composition to get a BC suspension. However, compared to a BC suspension—the BC powder composition as described herein may also be used for other commercially relevant purposes-such as a use e.g. only involving a relatively minor rehydration of the powder (i.e. not getting what the skilled person would understand to be a suspension).

As known in the art, and discussed in further detail below—the product of interest may be many different commercial relevant products of interest-such as e.g. a paper, a bag, a hair product, a cosmetic cream, etc.

Embodiments of the present invention are described below, by way of examples only.

### Bacterial Cellulose (BC) Powder-First Aspect

As discussed above-a first aspect of the present invention relates to a bacterial cellulose (BC) powder composition comprising:
(i): a BC: homogalacturonan (HG) ratio from 1:0.02 to 1:10000 (w/w); and
(ii): wherein the BC has a crystallinity index (CI) of 0.05 to 8, determined with Fourier

Transform Infrared spectroscopy (FT-IR) by calculating the peak ratio at 1430 and 898 cm⁻¹.

As discussed above-working Examples herein demonstrated that rhamnogalacturonan (RG) did not work as good as HG—i.e. one may say HG worked surprisingly good.

Accordingly, it may be preferred that the BC powder composition comprises a relatively low amount of RG.

Accordingly, in a preferred embodiment—the BC powder composition has a RG: BC ratio of less than 20:1 (wt:wt).

The term wt:wt and w/w are used interchangeably herein.

Preferably, the RG: BC ratio is lower than the HG: BC ratio. As understood by the skilled person in the present context in relation to this preferred embodiment-if e.g. the HG: BC ratio is 0.1:1, then the RG: BC ratio is lower than 0.1:1.

It is routine work for the skilled person to determine the amount of BC, HG and RG in a herein relevant (BC) powder composition of interest—i.e. it is routine work to determine the BC:HG and BC: RG ratios for a relevant (BC) powder composition of interest.

Preferably, the HG: BC ratio is from 0.03:1 to 100:1 (wt:wt); more preferably the HG: BC ratio is from 0.03:1 to 5:1 (such as e.g. from 0.05:1 to 5:1); even more preferably the HG: BC ratio is from 0.03:1 to 2:1 (such as e.g. from 0.05:1 to 2:1); and most preferably the HG: BC ratio is from 0.03:1 to 1.5:1 (such as e.g. from 0.03:1 to 5:1).

In some cases—e.g. for a specific BC powder of interest—it may be most preferred that the HG: BC ratio is from 0.07:1 to 0.15:1 (w/w).

Preferably, the RG: BC ratio is of less than 10:1 (w/w); more preferably RG: BC ratio is of less than 1:1 (w/w); even more preferably RG: BC ratio is of less than 0.01:1 (w/w); and most preferably the RG: BC ratio is of less than 0.001:1 (w/w).

Preferably, the BC: RG ratio is at least 5 times lower than the BC:HG ratio, more preferably the BC: RG ratio is at least 10 times lower than the BC:HG ratio, even more preferably the BC: RG ratio is at least 100 times lower than the BC:HG ratio, and most preferably the BC: RG ratio is at least 1000 times lower than the BC:HG ratio.

Preferably, the BC has a crystallinity index (CI) of 0.5 to 8; more preferably the BC has a crystallinity index (CI) of 0.75 to 8; even more preferably the BC has a crystallinity index (CI) of 1 to 8; and most the BC has a crystallinity index (CI) of 1.5 to 8.

For some practical reasons (see e.g. WO2022/200631A1 (Cellugy))—it may sometimes be difficult to obtain a crystallinity index (CI) above 7-accordingly, it may be preferred that the BC has a crystallinity index (CI) of 0.5 to 7; more preferably the BC has a crystallinity index (CI) of 0.75 to 7; even more preferably the BC has a crystallinity index (CI) of 1 to 7; and most the BC has a crystallinity index (CI) of 1.5 to 7.

In a preferred embodiment—the BC powder composition of the first aspect and and/or embodiments thereof (i.e. as described herein) comprises less carboxymethylcellulose (CMC) than HG (w/w); more preferably it comprises at least 10 times less CMC than HG, even more preferably it comprises at least 1000 times less CMC than HG; and most preferably it does not comprise CMC.

In a preferred embodiment—the BC powder composition of the first aspect and and/or embodiments thereof (i.e. as described herein) does not comprise isopropyl alcohol (IPA).

The BC powder composition may further comprise one or more further compounds, such as a water-soluble or a water-insoluble compound. The one or more further compounds may improve and/or change certain properties of the suspension, such as for example the stability, viscosity and/or the dispersibility of said suspension. The one or more further compounds may also be referred to as an additional component and/or additional components. Examples of such compounds include glycerin, ethylene glycol, dimethyl sulfoxide, dimethylformamide, lactic acid, gluconic acid and delta gluconolactone, a low molecular mass saccharide (e.g. glucose, fructose, galactose, xylose, etc.), salts (e.g. sodium sulfate, ammonium sulfate, sodium chloride, calcium chloride, etc.), amino acids, amino acid salts, acidic acids, organic acids, cation (e.g. Quaternary ammonium cation —QUAT), polyphenol, etc.

As discussed in working Example 13 herein—the positive recovery results of Examples 3 and 4 were not negatively affected by the addition of the cation (Quaternary ammonium cation-QUAT) additional component.

As known in the art (see e.g. WO2022/200631A1 (Cellugy))—bacteria which produce BC include for example those of the genus Acetobacter, Achromobacter, Aerobacter, Agrobacteria, Alcaligenes, Azotobacter, Gluconacetobacter, Gluconobacter, Komagataeibacter, Escherichia, Rhizobium, Pseudomonas, Salmonella and Sarcine—i.e. any of these bacteria may be used to obtain a herein relevant BC. In a preferred embodiment, the BC is obtained using a culture of bacteria comprising Komagataeibacter (preferably Komagataeibacter xylinus) and/or Gluconobacter (preferably Gluconobacter oxydans). In a working Example herein-BC was obtained using a culture of bacteria comprising Komagataeibacter xylinus. Accordingly—in a more preferred embodiment, the BC is obtained using a culture of bacteria comprising Komagataeibacter (preferably Komagataeibacter xylinus).

In Example 3 herein was measured viscosity upon resuspension of herein relevant examples of BC powder compositions of the first aspect. In a preferred embodiment—the BC powder composition has a viscosity upon resuspension at least 300 mPa's (such as from 300 mPa·s to 200000 mPa·s, more preferably from 400 mPa·s to 100000 mPa·s, even more preferably from 500 mPa's to 50000 mPa·s) at a shear stress of 1 s⁻¹ at 25° C. —measured by:
(I): the BC powder composition is added to 0.5% (w/w) in deionized water and mixing is carried out using high shear homogenizer, and then homogenized at 10300 rpm for 10 minutes to obtain a BC suspension;
(II): the viscosity of the BC suspension of step (I) is determined using dynamic rheology measurements and is carried out on a rheometer, where the geometry used is a 40 mm plate with the gap set to 1000 μm-before each measurement, the sample is allowed to rest for 1 minute at 25° C.—and the viscosity measurements is carried out at 25° C. under rotational movement measuring the viscosity at shear rate of 1 s⁻¹. In Example 3 herein was measured viscosity upon resuspension (i.e. viscosity of re-dispersed BC powder)—viscosity is preferably measured according to Example 3 herein.

### A Method for Producing a BC Powder-Second Aspect

As discussed above-a second aspect of the present invention relates to a method for producing a BC powder composition of the first aspect and/or embodiments thereof comprising the steps of:
(a): Obtaining a broth from a fermenter comprising BC with a crystallinity index (CI) of the first aspect and/or embodiment thereof;
(b): Purifying the BC from the broth of step (a);
(c): Optionally, contacting the purified BC of step (b) with a functionalization agent to thereby functionalizing the BC;
(d): Optionally, comminution of the BC from step (b) and/or step (c) to get a BC particle size of interest;
(e): Adding HG to the BC of step (b), step (c), and/or step (d) in an amount that gives a BC:HG ratio from 1:0.02 to 1:10000 (w/w);
(f): Drying the BC with added HG of step (e) to get a BC powder composition of the first aspect and/or embodiment thereof.

Based on the technical teaching herein and the common general knowledge (see e.g. WO2022/200631A1 (Cellugy)—the skilled person may routinely perform the individual steps of the method of the second aspect—i.e. may routinely perform the method of the second aspect as such. As known in the art—the optional comminution step (d) may be done by e.g. homogenization. If the comminution step (d) is used—the adding of HG step (e) may be done before, after or during the comminution step (d).

In relation to the adding HG to the BC of step (e)—the amount of added homogalacturonan relates to desired BC:HG ratio of interest. For example, a BC:HG ratio of 1:0.1 (w/w) may be obtained by adding 10 g HG to 100 g BC.

As discussed in a working Example herein-spray drying gave a fine and white powder which was more easily re-dispersible than with oven drying—i.e. oven drying works acceptable—but spray drying may be preferred. Accordingly, in a preferred embodiment is the drying step (f) done by spray drying.

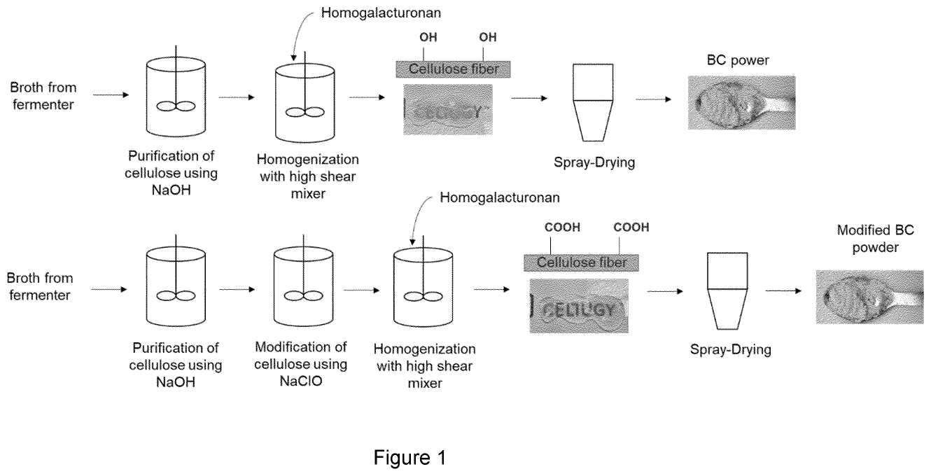

In a working Example herein was performed optional step (c) with functionalization agent NaClO (sodium hypochlorite) so accessible hydroxyl groups (—OH) were converted into carboxyl groups (—COOH)—se e.g. illustrative FIG. 1 herein. Accordingly, if the functionalizing the BC step (c) is done, the functionalization agent may be NaClO. Examples of suitable other known functionalization agents include e.g. nitric acid, acetic anhydride or TEMPO catalyst (see e.g. WO2022/200631A1 (Cellugy)).

### Use of BC Powder Composition for Making a Product of Interest-Use of Third Aspect

As discussed above-a third aspect of the present invention relates to a method to obtain a product of interest, wherein the method comprises a step of using a BC powder composition of the first aspect and/or embodiments thereof to obtain the product of interest and other suitable steps to obtain the product of interest.

The third aspect may alternatively be expressed as use of a BC powder composition of the first aspect and/or embodiments thereof for making a product of interest. As understood by the skilled person in the present context—the use of the third aspect to make a product of interest may involve a step of re-suspending the BC powder composition to get a BC suspension. However, compared to a BC suspension—the BC powder composition as described herein may also be used for other commercially relevant purposes-such as a use e.g. only involving a relatively minor rehydration of the powder (i.e. not getting what the skilled person would understand to be a suspension).

In a preferred embodiment of the method of the third aspect and/or embodiments thereof—the method comprises a step of re-suspending the BC powder composition to get a BC suspension—where is it preferred that the recovery of viscosity upon resuspension is at least 40% (preferably at least 50%, more preferably at least 55%, and most preferably at least 60%), measured at a shear stress of 1 s⁻¹ at 25° C. In Example 3 herein was measured recovery of viscosity upon resuspension-recovery of viscosity % is preferably measured according to Example 3 herein.

According to the art—the using of a BC powder composition as described herein to obtain the product of interest of the third aspect may involve a use of the BC powder composition:
(a): for coating a material, such as the surface of a material;
(b): as a particle stabilizer (particle stabilizator);
(c): as a emulsion stabilizer (e.g. emulsifier or co-emulsifier with or without mattifier capability);
(d): as a thickener (e.g. with or without anti-dripping property);
(e): as a rheology modifier; or
(f): as film former (e.g. antiwrinkle, anti-pollution, anti-photodamage).

Preferably, the BC powder composition is used as a thickener (e.g. to increase viscosity). It may be preferred that the method to obtain a product of interest is a method, wherein the step of using the BC powder composition of the first aspect and/or embodiments thereof to obtain the product of interest, comprises a step of rehydration of the BC powder composition by adding an amount of water to the BC powder composition that is so small that it does not create a BC suspension comprising more than 90% (w/w) of water and a BC suspension comprising more than 90% (w/w) of water is not created during any of the further suitable steps to obtain the product of interest.

More preferably, the step of rehydration of the BC powder composition is done by adding an amount of water to the BC powder composition that is so small that it does not create a BC suspension comprising more than 80% (w/w) of water, even more preferably does not create a BC suspension comprising more than 60% (w/w) of water, and most preferably does not create a BC suspension comprising more than 50% (w/w) of water.

Evidently and as understood by the skilled person in the present context-use of a “rehydration of the BC powder composition” step as discussed immediately above may in particular be preferred—wherein the product of interest is not a BC suspension as such (alternatively expressed as—with the proviso that the product of interest is not a BC suspension as such).

If the product of interest is e.g. a cream (e.g. a cosmetic cream)—it may be easier to control the final water content of the cream by a relatively minor rehydration of the powder—as compared to adding a BC suspension (comprising by itself a significant amount of water).

The term “cream” (e.g. a cosmetic cream and/or a skin cream) is understood by the skilled person to relate to e.g. the consistency of the cream product of interest—i.e. the skilled knows if a product of interest is a cream or not.

As known in the art—the product of interest may be many different commercial relevant products of interest-such as e.g. a BC suspension, a coating for a paper or a bag, a hair product, a cream, a gel, a lotion, a cosmetic (e.g. a cosmetic cream), a personal care product, a home care product, a textile, or a cellulose fiber-based material.

It may be preferred that the product of interest is a cream, cosmetic (e.g. a cosmetic cream) product or a personal care product.

In relation to a BC powder composition as described herein—it is not necessary to use CMC—e.g. due to the high improved recovery of viscosity as discussed herein. Accordingly, in a preferred embodiment of the method of the third aspect and/or embodiments thereof—the method does not comprise a step of adding carboxymethylcellulose (CMC) to a BC suspension obtained from the BC powder composition of the first aspect and/or embodiments thereof. It may be even more preferred, that the method does not comprise a step of adding CMC as such and the product of interest does not comprise CMC.

### Numbered Embodiments

1. A bacterial cellulose (BC) powder composition comprising:
(i): a BC: homogalacturonan (HG) ratio from 1:0.02 to 1:10000 (w/w); and
(ii): wherein the BC has a crystallinity index (CI) of 0.05 to 8, determined with Fourier Transform Infrared spectroscopy (FT-IR) by calculating the peak ratio at 1430 and 898 cm⁻¹.

2. The BC powder composition of embodiment 1, wherein the HG: BC ratio is from 0.05:1 to 5:1 (w/w).

3. The BC powder composition of embodiment 2, wherein the HG: BC ratio is from 0.07:1 to 0.15:1 (w/w).

4. The BC powder composition of any of the preceding embodiments, wherein the BC has a crystallinity index (CI) of 1.5 to 7.

5. The BC powder composition of any of the preceding embodiments, wherein the (BC) powder composition has a RG: BC ratio of less than 20:1 (w/w).

6. The BC powder composition of embodiment 5, wherein the (BC) powder composition has a RG: BC ratio of less than 0.001:1 (w/w).

7. The BC powder composition of any of the preceding embodiments, wherein the BC: RG ratio is lower than the BC:HG ratio.

8. The BC powder composition of embodiment 7, wherein the RG: BC ratio is at least 100 times lower than the HG: BC ratio.

9. The BC powder composition of embodiment 8, wherein the HG: BC ratio is the ratio of embodiment 2 and the BC has a crystallinity index of embodiment 4.

10. The BC powder composition of any of the preceding embodiments, wherein the BC is obtained using a culture of bacteria comprising Komagataeibacter, preferably Komagataeibacter xylinus.

11. The BC powder composition of any of the preceding embodiments, wherein the BC powder composition has a viscosity upon resuspension of at least 300 mPas at a shear stress of 1 s−1 at 25° C.—measured by:
(I): the BC powder composition is added to 0.5% (w/w) in deionized water and mixing is carried out using high shear homogenizer, and then homogenized at 10300 rpm for 10 minutes to obtain a BC suspension;
(II): the viscosity of the BC suspension of step (I) is determined using dynamic rheology measurements and is carried out on a rheometer, where the geometry used is a 40 mm plate with the gap set to 1000 μm-before each measurement, the sample is allowed to rest for 1 minute at 25° C.—and the viscosity measurements is carried out at 25° C. under rotational movement measuring the viscosity at shear rate of 1 s−1.

12. A method for producing a BC powder composition of any of the preceding embodiments comprising the steps of:
(a): Obtaining a broth from a fermenter comprising BC with a crystallinity index (CI) of any of the preceding embodiments;
(b): Purifying the BC from the broth of step (a);
(c): Optionally, contacting the purified BC of step (b) with a functionalization agent to thereby functionalizing the BC;
(d): Optionally, comminution of the BC from step (b) and/or step (c) to get a BC particle size of interest;
(e): Adding HG to the BC of step (b), step (c), and/or step (d) in an amount that gives a HG: BC ratio from 0.02:1 to 1000:1 (w/w);
(f): Drying the BC with added HG of step (e) to get a BC powder composition of any of the preceding embodiments.

13. A method to obtain a product of interest, wherein the method comprises a step of using a BC powder composition of any of the embodiments 1-11 to obtain the product of interest and other suitable steps to obtain the product of interest.

14. The method to obtain a product of interest of embodiment 13, wherein the product of interest is a BC suspension, a coating for a paper or a bag, a hair product, a cream, a gel, a lotion, a cosmetic (e.g. a cosmetic cream), a personal care product, a home care product, a textile, or a cellulose fiber-based material.

15. The method to obtain a product of interest of any of the embodiments 13-14, wherein the step of using the BC powder composition of any of the embodiments 1-11 to obtain the product of interest comprises a step of rehydration of the BC powder composition by adding an amount of water to the BC powder composition that is so small that it does not create a BC suspension comprising more than 90% (w/w) of water and a BC suspension comprising more than 90% (w/w) of water is not created during any of the further suitable steps to obtain the product of interest.

16. The method to obtain a product of interest of any of the embodiments 13-15, wherein the method does not comprise a step of adding carboxymethylcellulose (CMC) to a BC suspension obtained from the BC powder composition of any of the embodiments 1-11 and the method does not comprise a step of adding CMC as such and the product of interest does not comprise CMC.

### Examples

### Example 1-Method for Preparing BC Suspension Using Dynamic Fermentation Objective

To produce bacterial cellulose using a single isolate of Komagataeibacter xylinus (also known as Gluconobacter xylinus—these two names are used interchangeably herein), in an agitated reactor, method for purifying it and its end properties and performance.

### Materials and Methods

BC was synthesized in an agitated reactor using cellulose producing bacteria Komagataeibacter xylinus which was isolated by Cellugy from a symbiotic culture.
1. 10 mL of culture (inoculum) was mixed with 90 mL of HS (Hestrin & Schramm) medium with using sucrose as carbon source.
   1 L HS medium contained 5 g peptone, 5 g yeast extract, 1.15 g citrate, and 2.7 g disodium phosphate. The carbon sources used (per 1 l) were 20-50 g/L sucrose. All medium chemicals were supplied by Sigma Aldrich).
   The inoculum amount is 10% throughout all the production of BC in this application.
2. The culture was inoculated in HS medium in a standard agitated reactor for 72-120 H at room temperature (RT) (22±1° C.) with air flow rate of 0.2-0.4 vvm and agitator speed of 200-400 rpm. Sample for cellulose dry weigh evolution is taken every 24 H. The fermentation is stopped when there is no further increase in cellulose concentration measured with Anthrone method.
3. The BC was produced as finely dispersed cellulose cluster in the medium with cellulose content at range of 10-20 g/L dry weight.
4. The cellulose containing fermentation broth was then treated differently to see its performance in viscosity building and particle stabilization:
   a. Raw broth (used as is);
   b. Water-washed broth (washed with distilled water until conductivity of <1000 μS); or
   c. Purified cellulose (washed with 0.25 M NaOH and water in the end until conductivity <500 μS).

### Determination of Crystallinity

The crystallinity index (CI) or crystallinity % was determined with Fourier Transform Infrared spectroscopy (FT-IR) according to the method disclosed by O'Connor et al. (1958). The crystallinity index (CI) was determined by calculating the peak ratio at 1430 and 898 cm⁻¹.

### Determination if Viscosity of Raw (Untreated Broth)

Viscosity of produced cellulose in the fermenter during fermentation process was followed and determined using Anton Paar viscosimeter with following settings: RVT, 6 rpm, 1 min, 20° C., using spindle L3.

Determination of viscosity of raw, water-washed and purified BC broth (using rheometer) The viscosity measurements were carried out at 25° C. under rotational movement measuring the viscosity at shear rates between 0.1-10 s⁻¹ using a Discovery HR-20 Rheometer (TA Instruments). The geometry used was a 40 mm plate with the gap set to 1000 μm. The viscosity values presented are derived at a shear rate of 1 s⁻¹.

### Determination of Particle Stabilization

A total of 40 mg hydrogenated jojoba oil or jojoba beads (0.250-0.650 mm) were mixed into 10 mL of different concentrations of raw broth, water-washed BC broth and purified broth.

### Determination of Cellulose Content Using Anthrone Method

The cellulose content was measure using the Anthorne method which involves degradation of cellulose to sucrose using H₂SO₄, and measurement of absorbance at 620 nm using Anthrone in H₂SO₄.

### Results

Fermentation broth: cellulose containing fermentation was produced with a viscosity of 1500±100 mPas (Anton Paar) with a cellulose content of 15±2 g/L dry weight.

TABLE 1
Performance of differently treated BC suspensions.

| | | | | Viscosity | Minimum |
|---|---|---|---|---|---|
| | | Crystallinity | Viscosity | at 1.0 | dosage for |
| | | Measured | at 0.5 wt. % | wt. % | particle |
| | Conductivity | with FT-IR | (mPas at | (mPas at | stabilization |
| Product | (μS) | (%) | 1 s⁻¹) | 1 s⁻¹) | (wt. %) |
| Raw | >1,000 | 77 ± 1 | 207 | 662 | 1 |
| broth | | | (100-250) | (420-904) | |
| Water- | <1,000 | 77 ± 1 | 240 | 1,559 | 0.5 |
| washed | | | (203-270) | (1,287- | |
| BC broth | | | | 1,744) | |
| Purified | <500 | 77 ± 1 | 8,836 | 24,656 | 0.04 |
| BC broth | | | (2,998- | (13,815- | |
| | | | 18,499) | 35,921) | |

Crystallinity index related parameters of the BC produced with different carbon sources can be seen below.

TABLE 2
Crystallinity of BC produced in HS medium
using different concentration of sucrose.

| Sucrose in HS medium | Crystallinity index | Crystallinity % |
|---|---|---|
| 2-5% sucrose | 2-6 | 60-94 |
| 2% sucrose | 4.5 | 82 |

### Conclusion

Based on the viscosity and particle stabilization data, it was shown that purified BC has the best performance where it shows the highest viscosity and the lowest dosage to stabilize particle. Thus, purified BC suspension was used for the subsequent experiments and examples in this document.

### Example 2-Treatment of the Recovered BC to Produce the Powder

### Objective

Purification and drying of the BC.

FIG. 1 illustrates relevant steps for making non-modified and modified BC powder composition products.

The broth was obtained from a fermenter that had produced BC according to Example 1 where the purified cellulose of Table 1 of was used.

### Materials and Methods

### Purification of Cellulose Using NaOH

Broth from the fermenter was treated with 0.25 M NaOH at 60° C. to purify cellulose. The cellulose fraction was then collected/washed either using centrifugation or filtration.

### Modification of Cellulose Fiber Surface Using NaClO-Only for “Modified BC”

The purified cellulose was additionally treated with 0.5% NaClO prior to the addition of homogalacturonan to produce “modified BC” (in Examples 3 and 4), according to the method (Saito et al. 2007). This modification's objective is to have a stronger cellulose-water interaction by converting hydroxyl groups into carboxyl groups on the fiber surface.

Addition of Homogalacturonan (HG) in the Form of High HG Content Pectin and Homogenization with High-Shear Mixer

Pectin is composed of complex polysaccharides rich in galacturonic acid. There are three different domains of pectin referred to as homogalacturonan (HG), rhamnogalacturonan I (RG-I), and rhamnogalacturonan II (RG-II). Homogalacturonan is a linear homopolymer with the highest galacturonic acid content and without any side chains. RG I and II consists of a galacturonic acid backbone, with side chains comprising of neutral sugars. Across different plant cell structures, on average HG constitutes ca. 60-65% of the pectic fraction, with minor amount of RG-I (ca. 20-35%) and RG-II (ca. 10%) (Canteri et al., 2012).

The exact HG content may vary depending on extraction method and source of raw material. Throughout the examples, pectin with a relatively high HG content (at least 88% HG with 1-10% RGI and RGII) was used.

High HG content pectin was purchased from Sigma Aldrich (galacturonic acid ≥74.0%, methoxy groups ≥6.7%). Rhamnogalacturonan (RG) that was used for comparison (see results section) was purchased from Megazymes (purity >97%, galacturonic acid=51%).

Varying amounts of homogalacturonan was added to the BC in the form of high HG content pectin. The amount of added high HG content pectin relates, throughout the Examples to the desired BC:HG ratio of interest. For example, a BC:HG ratio of 1:0.1 (w/w) may be obtained by adding one weight unit high HG content pectin to ten weight units BC. In other words, throughout the Examples, the BC:HG ratio is considered equal to the BC: pectin ratio. Furthermore, throughout the Examples, “high HG content pectin” and “HG” is used synonymously.

The cellulose-homogalacturonan suspension was homogenized or mixed using a Silverson L5M mixer.

### Drying

The homogenous cellulose-homogalacturonan suspension was spray dried into a fine powder. In Example 3 and 4, a lab scale spray drier was used (Procept 4M8-Trix) whereas all other examples used a pilot scale spray drier (SiccaDania SD900). For comparison, instead of spray-drying, oven drying was also tested. Oven drying was performed at 60° C. for 24 hours and milling was carried out using a simple grinder.

### Results

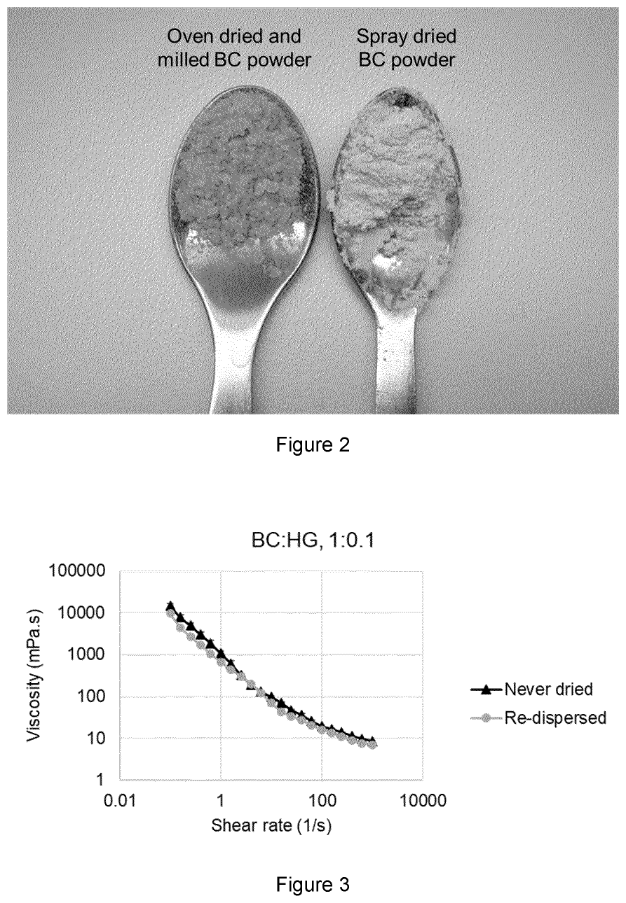

The BC powders were produced. The spray drying resulted in a fine and white powder which was more easily re-dispersible than with oven drying, i.e. oven drying works acceptable but spray drying may be preferred (see Table 3 and FIG. 2).

TABLE 3
The effect of different drying techniques on powder
dispersibility and visual appearance. The BC:HG ratio
was 1:0.1 (see Material and Methods of Example 2).

| Drying | Powder | Dispersion | Suspension |
|---|---|---|---|
| technique | appearance | technique | appearance |
| Spray-dried | Fine white | Silverson mixer | White gel |
| | powder | | |
| Oven-dried | Off-white, brown | Silverson | Off-white gel |
| | tinted, grainy | mixer, vortex, | with a brown |
| | powder | or propeller | tint |
| | | etc.* | |

*See Table 10 for details on dispersion techniques.

### Conclusions

The purified BC mixed with HG can preferably be spray dried to maximize visual appearance and lower redispersion energy.

### Example 3-Different Concentrations of HG and Comparison to RG: Viscosity Recovery of Redispersed BC Powder to Suspension

### Objective

To compare viscosity recovery of BC with different concentrations of HG and RG. The objectives are 1) to understand how much HG is needed, and 2) to highlight what type of pectin is needed to achieve BC redispersion (i.e. to investigate the importance of galacturonic acid content and structure).

### Material and Methods

Modified BC suspension with different HG concentrations were obtained according to Example 2. Thus, varying amounts of homogalacturonan in the form of high HG content pectin was added to the BC suspension. A BC:HG ratio of 1:0.1 (w/w) was for example obtained by adding ten weight units BC to one weight unit high HG content pectin. Drying was carried out with a lab scale spray drier.

For comparison, rhamnogalacturonan was added instead of homogalacturonan. As known in the art (see e.g. Zdunek et al. (Compr Rev Food Sci Food Saf. 2020; 1-17) pectin is a complex macromolecule obtained from the cell walls of plants and its different parts. Upon its extraction different parts may be obtained i.e., containing different amounts of homogalacturonan (HG), rhamnogalacturonan II (RG-II), and rhamnogalacturonan I (RG-I). Accordingly, and as known in the art, different commercially available pectin products may have different amounts of HG (as determined by the amount of galacturonic acid).

The BC powder formulation was added to 0.5% (w/w) in deionized water and mixing was carried out using a high shear Silverson L5M homogenizer with a tubular mixing unit Micro 5/8 “SL. The sample was homogenized at 10300 rpm for 5-10 minutes to obtain a re-dispersed powder.

The viscosity was determined using dynamic rheology measurements and were carried out on a Discovery HR-20 Rheometer (TA Instruments). The geometry used was a 40 mm plate with the gap set to 1000 μm. Before each measurement, the sample was allowed to rest for 1 minute at 25° C. The viscosity measurements were carried out at 25° C. under rotational movement measuring the viscosity at shear rates between 0.01-100 s⁻¹. The recovery of viscosity upon redispersion was calculated as follows:

$$Viscosity recovery ( \% ) = 100 * \frac{Viscosity ( at 1 s - 1 ) of redispersed BC powder}{Viscosity ( at 1 s - 1 ) of never dried BC suspension}$$

The storage modulus and loss modulus (G′ and G”) recovery (elasticity/gel strength) was determined by Dynamic rheology. Measurements were carried on a Discovery HR-20 Rheometer (TA Instruments). The geometry used was a 40 mm plate with the gap set to 1000 μm. Before each measurement, the sample was allowed to rest for 1 minute at 25° C. Oscillation sweeps were measured at 25° C. between strains of 0.01-100% at a frequency of 1 Hz.

### Results

The results are shown in Tables 4 and 5 below for single data point at shear rate 1 s⁻¹. For viscosity profile see FIG. 3.

TABLE 4
Viscosity recovery with different amount of HG in
the form of high HG content pectin (see Material
and Methods of Example 2) added to BC before drying.

| | Recovery of viscosity | Viscosity of re- |
|---|---|---|
| BC:HG | upon re-dispersion | dispersed (mPa · s |
| ratio | (%) | at 1 s⁻¹) |
| 1:0 | 3.4 | n.a. |
| 1:0.01 | 0.4 | 24 |
| 1:0.1 | 61.8 | 664 |
| 1:0.2 | 49.9 | 718 |
| 1:1 | 68.9 | 761 |

TABLE 5
Viscosity recovery with different amount
of RG added to BC before drying.

| | Recovery of viscosity | Viscosity of |
|---|---|---|
| BC:RG | upon re-dispersion | Re-dispersed |
| ratio | (%) | (mPa · s at 1 s⁻¹) |
| 1:0 | 3.4 | n.a. |
| 1:0.01 | 0.2 | 10 |
| 1:0.1 | 0.2 | 7 |
| 1:0.2 | 0.2 | 6 |
| 1:1 | 32.3 | 808 |

The fact that the BC:HG ratio 1:0.2 recovery (%) of 49.9 is lower than the ratios 1:0.1 and 1:1 may be explained as being related to some measurement uncertainty, i.e. the recovery (%) result of ratios 1:0.1, 1:0.2, and 1:1 may all be seen as relatively similar, all significantly better than BC:HG ratios 1:0 and 1:0.01.

A BC:HG ratio of 1:0.1 gave a very good re-dispersible BC powder with reduced loss of pre-drying characteristics (i.e. good recovery of viscosity upon redispersion). The results also demonstrated that RG did not work as good as HG.

### Conclusions

The results of this example show that BC powder with a BC:HG ratio of at least 1:0.1 to 1:0.2 may be considered preferred since it had a good recovery of viscosity upon resuspension (%).

A pectin grade such as RG (low galacturonic acid content) showed to be less effective in achieving a re-dispersible BC powder. The reason may be, as several studies indicate (previously cited), that homogalacturonan is closely associated with cellulose. Thus, HG (pectin with high galacturonic acid content) may bind to cellulose. This means that BC fibers gets “coated” with HG which works as an effective “spacer” at lower concentration to prevent hornification during the drying process.

### Example 4-Different Concentrations of HG and Comparison to RG: Storage Modulus (G′) Recovery of Redispersed BC Powder to Suspension

### Objective

Compare storage modulus, G′ and recovery (elasticity/gel strength) for different concentrations of HG and RG.

### Materials and Methods

Modified BC suspension with different HG concentrations were obtained according to Example 2. Thus, varying amounts of homogalacturonan in the form of high HG content pectin was added to the BC suspension. A BC:HG ratio of 1:0.1 (w/w) was for example obtained by adding ten weight units BC to one weight unit high HG content pectin. Drying was carried out with a lab scale spray drier.

For comparison, rhamnogalacturonan was added instead of homogalacturonan. As known in the art (see e.g. Zdunek et al. (Compr Rev Food Sci Food Saf. 2020; 1-17) pectin is a complex macromolecule obtained from the cell walls of plants and its different parts. Upon its extraction different parts may be obtained i.e., containing different amounts of homogalacturonan (HG), rhamnogalacturonan II (RG-II), and rhamnogalacturonan | (RG-I). Accordingly, and as known in the art, different commercially available pectin products may have different amounts of HG (as determined by the amount of galacturonic acid).

The BC powder formulation was added to 0.5% (w/w) in deionized water and mixing was carried out using a high shear Silverson L5M homogenizer with a tubular mixing unit Micro ⅝″ SL. The sample was homogenized at 10300 rpm for 5-10 minutes to obtain a re-dispersed powder.

Storage modulus and loss modulus (G′ and G″) recovery (elasticity/gel strength) was determined by Dynamic rheology. Measurements were carried on a Discovery HR-20 Rheometer (TA Instruments). The geometry used was a 40 mm plate with the gap set to 1000 μm. Before each measurement, the sample was allowed to rest for 1 minute at 25° C. Oscillation sweeps were measured at 25° C. between strains of 0.01-100% at a frequency of 1 Hz. The recovery of viscosity upon redispersion was calculated as follows:

$${G}^{′} recovery ( \% ) = 100 * \frac{{G}^{′} ( at 1 hz ) of redispersed BC powder}{{G}^{′} ( at 1 hz ) of never dried BC suspension}$$

### Results

The results are shown in the tables below.

TABLE 6
Storage modulus (G′) recovery with different amount
of HG in the form of high HG content pectin (see Material
and methods of Example 2) added to BC before drying.

| | Recovery of | |
|---|---|---|
| | storage modulus | Storage modulus |
| BC:HG | upon redispersion | of re-dispersed |
| ratio | (%) | (Pa at 1% strain) |
| 1:0 | 0.8 | 1.13 |
| 1:0.01 | 0.1 | 0.10 |
| 1:0.1 | 47.4 | 6.62 |
| 1:0.2 | 44.2 | 7.61 |
| 1:1 | 51.3 | 6.62 |

TABLE 7
Storage modulus (G′) recovery with different
amount of RG added to BC before drying.

| | Recovery of | |
|---|---|---|
| | storage modulus | Storage modulus |
| BC:RG | upon redispersion | of re-dispersed |
| ratio | (%) | (Pa at 1% strain) |
| 1:0 | 0.8 | 1.13 |
| 1:0.01 | 0.1 | 0.06 |
| 1:0.1 | 0.0 | 0 |
| 1:0.2 | 0.0 | 0 |
| 1:1 | 29.8 | 9.06 |

The fact that the BC:HG ratio 1:0.2 recovery (%) of 44.2 is lower than the ratios 1:0.1 and 1:1 may be explained as being related to measurement uncertainty—i.e. the recovery (%) result of ratios 1:0.1, 1:0.2, and 1:1 may all be seen as relatively similar-all significantly higher than BC:HG ratios of 1:0 and 1:0.01.

FIG. 4 shows the storage modulus (G′) and loss modulus (G″) profiles of the BC powder with a BC:HG ratio of 1:0.1 measured as “Never dried” and “Re-dispersed”. A BC:HG ratio of 1:0.1 resulted in a highly re-dispersible BC powder with reduced loss of pre-drying characteristics (i.e. good recovery of elasticity/gel strength upon resuspension). The fact that G′ >G″ between strains of 0.01-10% shows that both “Never dried” and “Re-dispersed” suspensions behaved as gels.

### Conclusion

The results of this example show that BC powder with a BC:HG ratio of 1:0.1 as well as BC powders with higher ratios/concentrations of HG had a good recovery of G′ upon resuspension (%) (i.e. good recovery of elasticity/gel strength).

The storage and loss modulus profiles of a BC powder with a BC:HG ratio of 1:0.1 were measured as “Never dried” and “Re-dispersed”. A BC:HG ratio of 1:0.1 resulted in a highly re-dispersible BC powder with reduced loss of pre-drying characteristics (i.e. good recovery of elasticity/gel strength upon resuspension).

The results further demonstrated that BC powders with RG were not as re-dispersible as BC powders with HG.

### Example 5-Higher Viscosity and G′ Recovery with Non-Modified BC Used for Powder Production (BC:HG Ratio 1:1)

### Objective

To demonstrate the effectiveness of HG when used with non-modified BC and a high HG amount.

### Materials and Methods

BC suspensions with different HG concentrations were obtained according to Example 2. Thus, varying amounts of homogalacturonan in the form of high HG content pectin was added to the BC suspension. A BC:HG ratio of 1:0.1 (w/w) was for example obtained by adding ten weight units BC to one weight unit high HG content pectin. Drying was carried out with a pilot scale spray drier.

Viscosity and storage modulus recovery tests were repeated according to Examples 3 and 4 above for a BC powder that comprises non-modified cellulose.

### Results

Viscosity recovery and storage modulus recovery were repeated according to Example 3 and 4 above for a BC powder (1.6% in water) with a BC:HG ratio of 1:1 that comprises non-modified cellulose.

The results were positive—i.e. the HG seems to have even better effect on re-dispersibility of non-modified BC. See FIG. 5 for comparison between never-dried and re-dispersed BC formulation.

TABLE 5
Viscosity recovery with BC:HG ratio of 1:1 (see Materials and methods
of Example 2) after re-dispersion of the powder formula.

| | Recovery of viscosity | Viscosity of |
|---|---|---|
| BC:HG | upon re-dispersion | re-dispersed |
| ratio | (%) | (mPa · s at 1 s⁻¹) |
| 1:1 | 97 | 23,400 |

TABLE 5
Viscosity recovery with BC:HG ratio of 1:1 (see Materials and methods
of Example 2) after re-dispersion of the powder formula.

| | Recovery of storage | Storage modulus of |
|---|---|---|
| BC:HG | modulus upon re-dispersion | re-dispersed |
| ratio | (%) | (Pa at 1% strain) |
| 1:1 | 100 | 606.7 |

### Conclusion

With non-modified BC, the re-dispersibility on the BC powder was further improved in terms of viscosity and G′ recovery.

### Example 6-Higher Viscosity, G′, and Water Holding Recovery with Non-Modified BC Used for Powder Production (BC:HG Ratio 1:0.1 and 1:0.2)

### Objective

To demonstrate the effectiveness of HG when used with non-modified BC and lower HG amount.

### Materials and Methods

BC suspensions with different HG concentrations were obtained according to Example 2. Thus, varying amounts of homogalacturonan in the form of high HG content pectin was added to the BC suspension. A BC:HG ratio of 1:0.1 (w/w) was for example obtained by adding ten weight units BC to one weight unit high HG content pectin. Drying was carried out with a pilot scale spray drier.

Viscosity recovery tests were repeated according to Example 3 above for a BC powder that comprises non-modified cellulose.

Water holding capacity was additionally tested and compared between the never dried and re-dispersed BC suspensions. The BC suspension (20 mL of 0.5% BC in deionized water) was centrifuged at 4500×G for 20 minutes. The supernatant was carefully removed and weighed. The water holding capacity was calculated based on how much water the cellulose pellet absorbed using the following equation where Wis weight.

$$( {W}_{total} - {W}_{supematant} - {W}_{drypellett} ) / {W}_{drypellett}$$

### Results

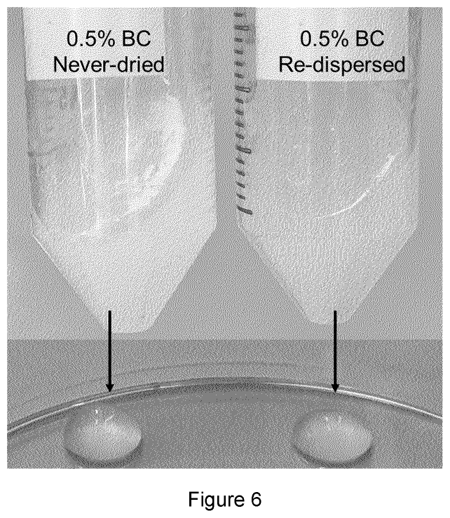

The results were positive, i.e. the HG seems to have even better effect on re-dispersibility of non-modified BC, especially with a BC:HG ratio of 1:0.2. See Tables 6-7, as well as FIG. 6 which shows the dispersed powder suspension.

TABLE 6
Viscosity recovery with different amount of HG in
the form of high HG content pectin (see Material
and Methods of Example 2) added to BC before drying.

| | Recovery of viscosity | Viscosity of |
|---|---|---|
| BC:HG | upon re-dispersion | re-dispersed |
| ratio | (%) | (mPa · s at 1 s⁻¹) |
| 1:0.1 | 66 | 8,150 |
| 1:0.2 | 96 | 11,815 |

TABLE 7
Storage modulus recovery with different amount of HG
in the form of high HG content pectin (see Material
and methods of Example 2) added to BC before drying.

| | Recovery of storage | Storage modulus |
|---|---|---|
| BC:HG | modulus upon re-dispersion | of re-dispersed |
| ratio | (%) | (Pa at 1% strain) |
| 1:0.1 | 63 | 186 |
| 1:0.2 | 85 | 248 |

TABLE 8
Recovery of water holding capacity with different amount
of HG in the form of high HG content pectin (see Material
and methods of Example 2) added to BC before drying.

| | Recovery of WHC | |
|---|---|---|
| BC:HG | upon redispersion | WHC of re-dispersed |
| ratio | (%) | (g water/g BC) |
| 1:0.1 | 90 | 147 |
| 1:0.2 | 100 | 158 |

### Conclusion

With non-modified BC, the re-dispersibility of the BC powder was further improved in terms of viscosity recovery, G′ recovery, and water holding capacity.

### Example 7-Spray Dried BC Powder Characteristics

### Objective

To characterize the size and morphology of particles comprising the spray-dried BC powder formulation.

### Materials and Methods

A BC:HG mixture was prepared according to Example 2. Thus, varying amounts of homogalacturonan in the form of high HG content pectin was added to the BC suspension. A BC:HG ratio of 1:0.15 (w/w) was obtained by adding one weight unit BC to 0.15 weight units high HG content pectin. The BC:HG mixture was spray dried into a powder. The chosen BC:HG ratio was 1:0.15 for maximizing re-dispersibility while maintaining a low HG content. Drying was carried out with a pilot scale spray drier.

Morphological analysis was performed using a Malvern Morphology G3 microscope, SN: MAL 1016763. Morphology G3 microscope is an automated light microscope applied with a camera, allowing characterization of thousands of particles in a short period of time. By applying statistical calculation on the large dataset, the following parameters were determined: circularity, convexity, and elongation. Furthermore, the average particle size, referred to as circular equivalent (CE) diameter, was determined. All parameters can either be calculated based on number distribution (ND), where all particles are weighed equally, or volume distribution (VD), where each particle is weighed based on their volume meaning smaller particles contribute less to the average than larger particles.

### Results

See Table 6. The number distribution (ND) is used to determine the properties of all the particles in a sample, however, this is often not correlated to the properties of the powder, as the larger particles have a bigger influence on the properties of the powder than smaller particles. Thus, the parameters determined by VD are more suitable for describing the properties of a powder.

The CE-diameters for all samples are in the same range both when determined based on ND and VD. By comparing the CE-diameters based on ND with the ones based on VD, a 3 to 5-fold increase is observed. This indicates that there are particles with small sizes in the samples, but their contribution is low compared to bigger particles in the samples. Hence, the particle sizes of the powders are better described as being between 19 to 24 μm.

For all samples, the circularity is larger when determined based on VD. The circularity of all samples is close to 0.9, which means that the particles in the powders have a circular shape.

The convexity does not differ based on ND or VD, and for all samples a convexity close to 1 is observed. This means that each sample is composed of particles with a smooth surface.

A doubling in elongation is observed for all values determined by VD compared to ND. The VD values for the four samples are in the range of 0.50 to 0.62, which are high elongation values.

TABLE 9
Morphological particle characterization of sample 1-4
which correspond to different batches of the BC powder.

| | CE-diameter (μm) | | | | | | | | | | |
|---|---|---|---|---|---|---|---|---|---|---|---|
| | | | D[n, | D[n, | D[n, | Circularity | | Convexity | | Elongation | |
| Batch | ND | VD | 10] | 50] | 90 | ND | VD | ND | VD | ND | VD |
| 1 | 6.98 | 20.75 | 3.43 | 5.88 | 11.61 | 0.816 | 0.879 | 0.972 | 0.978 | 0.272 | 0.551 |
| 2 | 7.40 | 23.71 | 3.60 | 6.28 | 12.25 | 0.817 | 0.880 | 0.972 | 0.978 | 0.271 | 0.571 |
| 3 | 5.91 | 19.01 | 2.50 | 5.06 | 9.94 | 0.868 | 0.906 | 0.987 | 0.989 | 0.249 | 0.611 |
| 4 | 4.47 | 20.52 | 1.75 | 3.50 | 8.28 | 0.836 | 0.894 | 0.978 | 0.982 | 0.243 | 0.501 |

### Conclusion

The powder can be described as elliptical-shaped particles with a smooth surface and an average size of 19-24 μm. This can be considered a small particle size with a narrow size distribution which facilitates re-dispersibility of the powder (i.e. lowering the energy input needed for dispersion).

### Example 8-Dispersibility of BC Powder Using Different Redispersion Techniques

### Objective

To test viscosity recovery of the BC powder formulation using different mixing techniques.

### Materials and Methods

BC suspensions were obtained according to Example 2. Viscosity recovery was repeated according to Example 4 for a BC powder with a BC:HG ratio of 1:0.15. The BC:HG was obtained by adding one weight unit of BC to 0.15 weight units of high HG content pectin The given BC:HG ratio was chosen for maximizing re-dispersibility while maintaining a low HG content. Drying was carried out with a pilot scale spray drier.

Different powder dispersion techniques were tested, for example, with Ultra-turrax (10,000 rpm) for 2 minutes and Vortex shaking (5000 rpm) for 5 minutes.

### Results

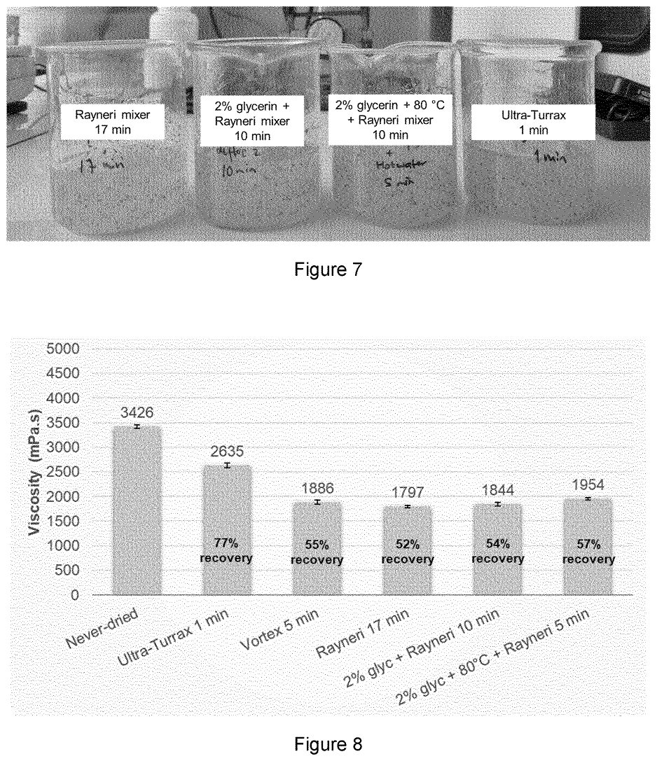

A broad range of mixing techniques were effective for dispersing the BC powder into a stable suspension. All resulting suspensions were able to stabilize 250-650 μm beads over at least several weeks (FIG. 7). High-shear mixers such as Ultra-Turrax or Silverson was most effective for dispersing the BC powder in terms of viscosity recovery. Nonetheless, zero-shear mixing with e.g. vortex/shaking, Rayneri mixer, and propeller was sufficient (FIG. 8). Additionally, pre-wetting the powder in glycerine and/or heating to 80° C. was shown to lower the re-dispersion time with zero-shear mixer. Table 10 shows examples of dispersion techniques that were found to be effective to disperse the BC powder into a stable suspension with a minimum of 50% viscosity recovery.

TABLE 10
Different mixing techniques for dispersing the BC powder in water.

| | | | Dispersion | | Final |
|---|---|---|---|---|---|
| | Speed | Batch size | time | | conc. |
| Dispersion equipment | (rpm) | (liter) | (min) | | (%) |
| Ultra-Turrax | 10,000 or | 0.02-0.2 | 1-2 | | 1-2 |
| | max | | | | |
| Vortex/shaking | 5,000 | 0.02-0.2 | 5 | | 1-2 |
| Silverson homogenizer | 8,000 or | 0.2-10 | 2-5 | | 1-2 |
| | max | | | | |
| Propeller | <3,000 | Up to | >20 | min | 1-2 |
| | | 1000 | | | |
| Rayneri mixer/deflocculator | 600-1000 | 0.2-20 | 17 | min | 1-2 |
| Rayneri/deflocculator + | 600-1000 | 0.2-20 | 10 | min | 1-2 |
| pre-wetting with glycerin | | | | | |
| Rayneri/deflocculator + | 600-1000 | 0.2-20 | 5 | min | 1-2 |
| pre-wetting with glycerin + | | | | | |
| heating 80 C. | | | | | |

### Conclusion

The BC powder can be dispersed with high-shear mixing (Silverson or Ultra-Turrax) as well as conventional mixing (vortex, propeller, or Rayneri) and retains at least 50% of the viscosity relative to a never-dried BC suspension.

### Example 9-Viscosity of BC Powder

### Objective

To measure the viscosity of the BC powder formulation at different shear rates and different concentrations.

### Materials and Methods

BC suspensions were obtained according to Example 2. A BC powder with a BC:HG ratio of 1:0.15 was obtained by adding one weight unit of BC to 0.15 weight units of high HG content pectin. The given BC:HG ratio was chosen for maximizing re-dispersibility while maintaining a low HG content. Drying was carried out with a pilot scale spray drier.

The BC powder was dispersed in water at 0.04, 0.125, 0.25, 0.5, and 1.0 wt. % using Ultra-Turrax mixing (10 000 rpm) for 2 minutes.

The viscosity was determined using dynamic rheology measurements and were carried out on a Discovery HR-20 Rheometer (TA Instruments). The geometry used was a 40 mm plate with the gap set to 1000 μm. Before each measurement, the sample was allowed to rest for 1 minute at 25° C. The viscosity measurements were carried out at 25° C. under rotational movement measuring the viscosity at shear rates between 0.01-100 s−1. For determining viscosity at different BC concentrations, the viscosity value at a shear rate of 1 s⁻¹ was selected.

### Results

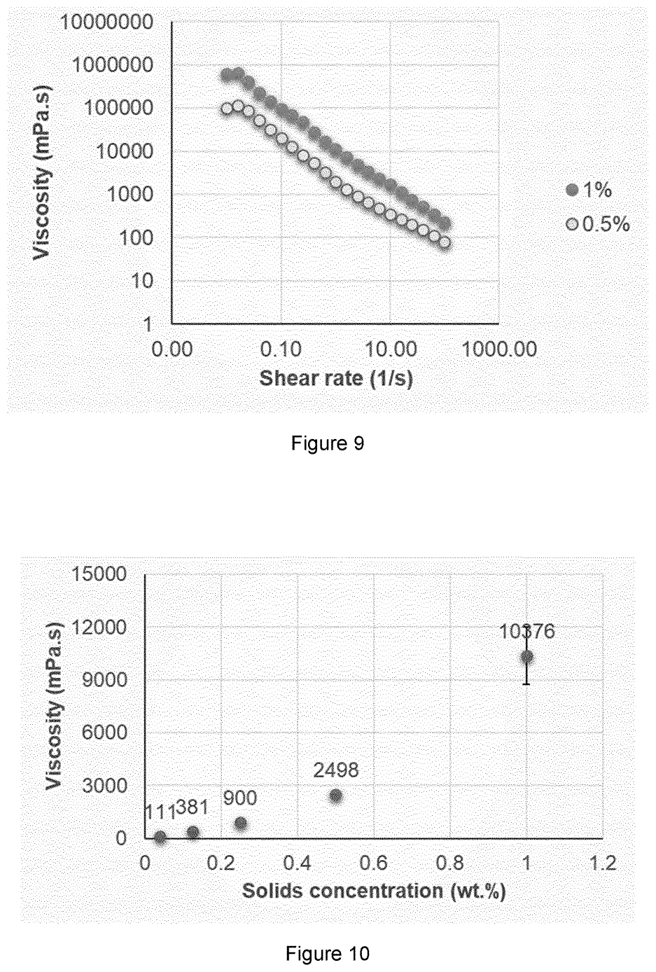

FIG. 9 shows a shear thinning behaviour and stable profile of the re-dispersed BC powder at concentrations such as 0.5 and 1%. The viscosity at different concentrations of the re-dispersed BC powder was also tested, see Table 11 and FIG. 10.

TABLE 11
Viscosity at different concentration of re-
dispersed BC powder formulation in water; the
standard deviation is shown within brackets.

| BC powder concentration in water | Viscosity at a shear rate of 1 s−1 | |
|---|---|---|
| (wt. %) | (mPa · s) | |
| 0.04 | 111 | (±13) |
| 0.125 | 381 | (±13) |
| 0.25 | 900 | (±57) |
| 0.5 | 2,498 | (±62) |
| 1.0 | 10,376 | (±1639) |

### Conclusion

The dispersed BC powder behaves identical to the never-dried BC suspension, i.e. shear thinning and linear-to-exponential increase in viscosity at increasing powder concentrations between 0.04-1.0 wt. % in water.

### Example 10-Particle Suspending Ability and Stability of Redispersed BC Powder to Suspension

### Objective

To test the particle suspending ability and stability over time of the dispersed BC powder.

### Materials and Methods

BC suspensions were obtained according to Example 2. Thus, varying amounts of homogalacturonan in the form of high HG content pectin was added to the BC suspension. A BC:HG ratio of 1:0.1 (w/w) was for example obtained by adding ten weight units of BC to one weight unit of high HG content pectin. Drying was carried out with a pilot scale spray drier.

The BC powder (with a BC:HG ratio of 1:0.1 and 1:0.2) was added up to a concentration of 0.05 wt. % in deionized water and dispersed using Ultra-Turrax mixing (10 000 rpm) for 2 minutes. Finally, jojoba beads (250-650 μm) were added to a concentration of 0.4 wt. %.

### Results

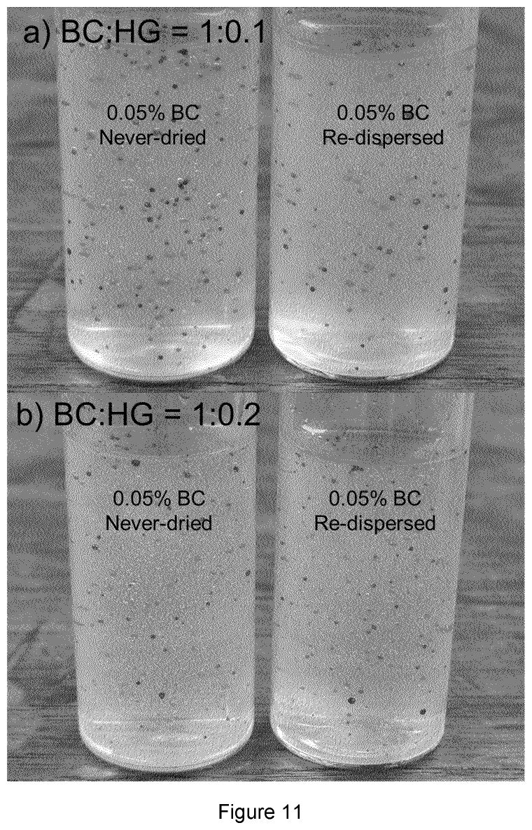

The resulting BC suspensions were stable, i.e., no cellulose-water separation or sedimentation of suspended particles was observed over at least 6 months (Table 12 and FIG. 11).

TABLE 12
Particle stability in never dried and re-dispersed BC suspensions.

| | BC:HG = 1:0.1 | | BC:HG = 1:0.2 | |
|---|---|---|---|---|
| | Never dried | Re-dispersed | Never dried | Re-dispersed |
| Particle | BC | BC | BC | BC |
| stability | (0.05 wt. %) | (0.05 wt. %) | (0.05 wt. %) | (0.05 wt. %) |
| Over 6 | Stable | Stable | Stable | Stable |
| months | | | | |

### Conclusion

The BC powder results in a stable suspension upon dispersion which can be used to suspend beads at as low dosage as in the never-dried BC suspension.

### Example 11-pH Stability of Redispersed BC Powder to Suspension

### Objective

To demonstrate pH stability of the re-dispersed BC powder formulation.

### Materials and Methods

BC suspensions were obtained according to Example 2. A BC powder with a BC:HG ratio of 1:0.15 was obtained by adding one weight unit of BC to 0.15 weight units of high HG content pectin. The given BC:HG ratio was chosen for maximizing re-dispersibility while maintaining a low HG content. Drying was carried out with a pilot scale spray drier.

The BC powder was added to 1 wt. % in deionized water and dispersed using Ultra-Turrax mixing (10 000 rpm) for 2 minutes. Subsequently, the pH was adjusted by diluting the sample to 0.5 wt. % with a mixture of NaOH and citric acid.

### Results

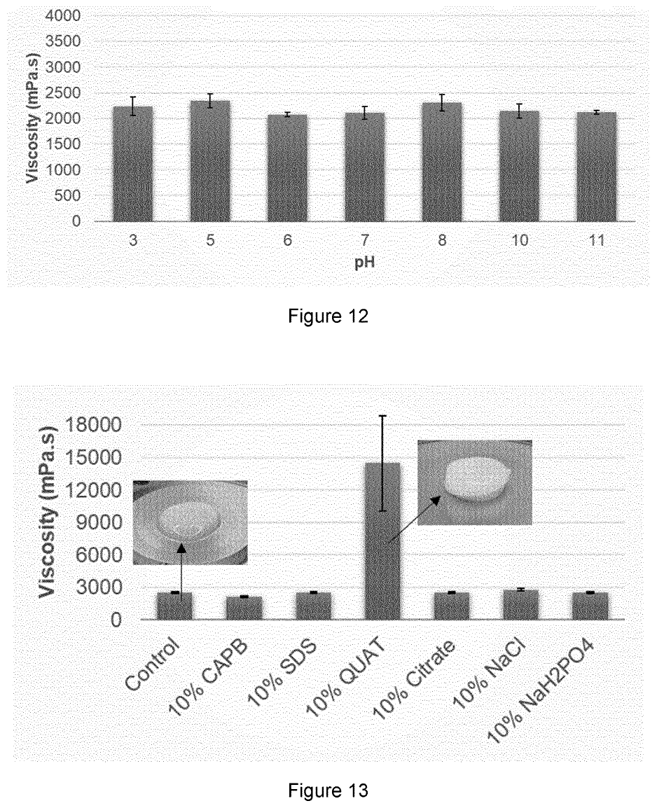

As seen in Table 13 and FIG. 12, the re-dispersed BC powder formulation is stable (i.e. viscosity does not change) across different pH.

TABLE 13
The viscosity of 0.5 wt. % re-dispersed BC powder
formulation in water at different pH; the standard
deviation is shown within brackets.

| pH | Viscosity at a shear rate of 1 s−1 (mPa · s) | |
|---|---|---|
| 3 | 2,238 | (±185) |
| 5 | 2,347 | (±136) |
| 6 | 2,074 | (±45) |
| 7 | 2,112 | (±126) |
| 8 | 2,307 | (±165) |
| 10 | 2,147 | (±136) |
| 11 | 2,121 | (±38) |

### Conclusion

Due to the low amount of HG needed to obtain a re-dispersible BC powder, the mixture retains its stability/viscosity across different pH.

### Example 12-Salt and Surfactant Stability of Redispersed BC Powder to Suspension

### Objective

To demonstrate surfactant and salt stability of the dispersed BC powder formulation.

### Materials and Methods

BC suspensions were obtained according to Example 2. A BC powder with a BC:HG ratio of 1:0.15 was obtained by adding one weight unit of BC to 0.15 weight units of high HG content pectin. The given BC:HG ratio was chosen for maximizing re-dispersibility while maintaining a low HG content. Drying was carried out with a pilot scale spray drier.

The BC powder was added to 0.5 wt. % in deionized water and dispersed using Ultra-Turrax mixing (10 000 rpm) for 2 minutes. Different surfactants or salts were added to the BC suspension to final concentration of 10 wt. %.

### Results

As seen in Table 14 and FIG. 13, the redispersed BC powder formulation is stable in the presence of zwitter ionic surfactant (Cocamidopropyl Betaine, CAPB), anionic surfactant (Sodium dodecyl sulfate, SDS), and cationic surfactant (Quaternary ammonium compound, QUAT) as well as different salts. There was a surprising synergy between the BC powder formulation and QUAT resulting in a stiff gel and 5-fold increase in viscosity.

TABLE 14
The viscosity of 0.5 wt. % re-dispersed BC powder formulation
in water with different surfactants and salts; the
standard deviation is shown within brackets.

| Surfactant or salt | Viscosity at a shear rate of 1 s−1 | |
|---|---|---|
| (10 wt. %) | (mPa · s) | |
| Control | 2,541 | (±79) |
| Cocamidopropyl Betaine | 2,114 | (±50) |
| Sodium dodecyl sulfate | 2,512 | (±62) |
| Quaternary ammonium compound | 14,465 | (±4430) |
| Citrate | 2,532 | (±78) |
| NaCl | 2,759 | (±105) |
| NaH₂PO₄ | 2,519 | (±49) |

### Conclusion

Due to the small amount of additive (HG) needed to obtain a re-dispersible BC powder, the mixture retains its viscosity and is stable in the presence of different surfactants and salts.

### Example 13-Formulation Example: Cleanser Gel with BC Powder Objective

To demonstrate the use of BC powder in a “challenging” formulation, i.e. in conditions where various thickening agents may not work.

### Materials and Methods

BC suspensions were obtained according to Example 2. A BC powder with a BC:HG ratio of 1:0.15 was obtained by adding one weight unit of BC to 0.15 weight units of high HG content pectin. The given BC:HG ratio was chosen for maximizing re-dispersibility while maintaining a low HG content. Drying was carried out with a pilot scale spray drier. Following steps were carried out to make the cleanser formulation:
1. Prepare buffer solution.
2. Prepare phase A:
   a) Weigh up BC powder based on the final concentration.
   b) Mix DI-water and glycerine.
   c) Put mixture of DI-water and glycerine in Ultra-Turrax/Silverson at 8000-10 000 rpm and add slowly BC powder until all is incorporated. Continue mixing for 1-2 mins (for 50-250 mL scale).
   d) Degass the mixture if foaming formed.
3. Prepare phase B:
   a) Mix both surfactant types using magnetic bar for 5-10 mins.
4. Add phase B to phase A using magnetic bar mixer.
5. Add phenoxyethanol or preservative of your choice.
6. Adjust the pH of the mixture using phase C component to pH 5.

TABLE 15
Ingredient list for cleanser formulation.

| | | Gel cleanser | Gel cleanser |
|---|---|---|---|
| | Ingredients | no. 1 (wt. %) | no. 2 (wt. %) |
| Phase A | BC powder | 0.5 | 1 |
| (thickener/stabilizer) | DI-water | 80.85 | 80.35 |
| | Glycerin | 5 | 5 |
| Phase B | Anionic surfactant | 2 | 2 |
| (Surfactant) | (Caprylic glucoside) | | |
| | Amphoteric | 10 | 10 |
| | surfactant | | |
| | (Cocamidoprophyl | | |
| | betaine) | | |
| Phase C | 50% citric acid | To pH 5 | To pH 6 |
| (Buffer) | 1M NaOH | | |
| Preservative | Phenoxyethanol | 0.5 | 0.5 |

### Results

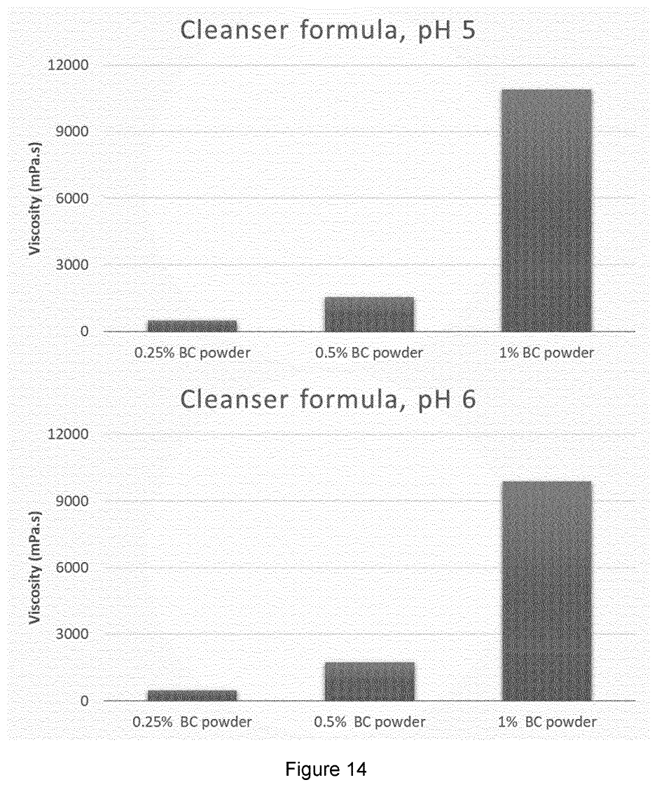

The dispersed BC powder formulation retained its viscosity in the cleanser formulations, i.e. in presence of salts and surfactants at low pH (Table 16 and FIG. 14).

TABLE 16
Viscosity of cleanser formulation containing surfactants
and low pH with different amount of BC powder added.

| BC powder (wt. %) in | | Viscosity (mPa · s) at a |
|---|---|---|
| cleanser formulation | pH | shear rate of 1 s−1 |
| 0.25 | 5 | 493 |
| 0.5 | 5 | 1,551 |
| 1.0 | 5 | 10,910 |
| 0.25 | 6 | 468 |
| 0.5 | 6 | 1,752 |
| 1.0 | 6 | 9,893 |

### Conclusion

Due to the low amount of additive (HG) needed to achieve a re-dispersible BC powder formulation, the dispersed material remains stable in challenging conditions such as low pH in combination with surfactants.

### REFERENCES

ZDUNEK, Artur; PIECZYWEK, Piotr M.; CYBULSKA, Justyna. The primary, secondary, and structures of higher levels of pectin polysaccharides. Comprehensive Reviews in Food Science and Food Safety, 2021, 20.1:1101-1117.
WO 2006/127810, Yang, Bacterial cellulose-containing formulations and method of producing effective bacterial cellulose-containing formulations, 2006.
WO 2020/136629 A1, Fernando Octávio QUEIRÓS DOURADO, Francisco Miguel Portela Da Gama, Bacterial cellulose formulations methods and uses thereof, 2020.
WO 2023/064733 A1, Mohammed Shafi SYED, Larry Rubic, Michelle Terese HOARD, Timothy OTJENS, Bastian ANDERSEN, Dispersible bacterial cellulose formulations, 2023.
JP 2873927 B2 [symbol] . Drying method and dried product of bacterial cellulose, 1995.
WO 2001/005838 A1, Zhi-Fa Yang, Sanjeev Sharma, Chat Mohan, Joseph Kobzeff, Process for drying reticulated bacterial cellulose without co-agents.
HIASA, Shou, et al. Prevention of aggregation of pectin-containing cellulose nanofibers prepared from mandarin peel. Journal of Fiber Science and Technology, 2016, 72.1:17-26.
HIETALA, Maiju; SAIN, Sunanda; OKSMAN, Kristiina. Highly re-dispersible sugar beet nanofibers as reinforcement in bionanocomposites. Cellulose, 2017, 24.5:2177-2189.
YAPO, Beda Marcel, et al. Pectins from citrus peel cell walls contain homogalacturonans homogenous with respect to molar mass, rhamnogalacturonan 1 and rhamnogalacturonan II. Carbohydrate Polymers, 2007, 69.3:426-435.
CANTERI, Maria Helene Giovanetti, et al. Characterization of apple pectin-a chromatographic approach. Chromatography—The most versatile method of chemical analysis, 2012, 325.

1 . A bacterial cellulose (BC) powder comprising:
a. BC; and
b. homogalacturonan (HG);
wherein the ratio of HG: BC is at least 0.02:1 (wt:wt).

2. The BC powder according to claim 1, wherein the HG: BC ratio is at least 0.05:1, such as at least 0.1:1, such as at least 0:15:1, such as at least 0.2:1, such as at least 0.5:1, such as at least 1:1.

3. The BC powder according to any one of the preceding claims, wherein the HG: BC ratio is at most 50:1, such as at most 20:1, such as at most 10:1, such as at most 5:1, such as at most 2:1.

4. The BC powder according to any one of the preceding claims, wherein the HG: BC ratio is between 0.02:1 and 50:1, such as between 0.05:1 and 20:1, such as between 0.05:1 and 10:1, such as between 0.1:1 and 10:1, such as between 0.1:1 and 5:1, such as between 0.1:1 and 1:1, such as between 0.1:1 and 0.5:1.

5. The BC powder according to any one of the preceding claims, wherein the BC has a crystallinity of at least 30%, such as at least 40%, such as at least 50%, such as at least 60%, such as at least 70%, such as at least 80%, wherein the crystallinity is determined with Fourier Transform Infrared spectroscopy (FT-IR) by calculating the peak ratio at 1430 and 898 cm⁻¹.

6. The BC powder according to any one of the preceding claims, wherein the BC has a crystallinity of between 30% and 100%, such as between 50% and 100%, such as between 40% and 90%, such as between 60% and 90%, such as between, 70% and 90%, such as between 70% and 85%, wherein the crystallinity is determined with FT-IR by calculating the peak ratio at 1430 and 898 cm⁻¹.

7. The BC powder according to any one of the preceding claims, wherein the BC powder comprises powder particles with:
a. an average powder particle size of between 0.1 and 500 μm, such as between 1 and 50 μm, such as between 10 and 40 μm, such as between 15 and 30 μm, such as between 19 and 24 μm,
wherein the average powder particle size is calculated based on the powder particle volume distribution;
b. an average powder particle size of between 0.01 and 200 μm, such as between 0.1 and 20 μm, such as between 1 and 15 μm, such as between 1 and 10 μm, such as between 2 and 9 μm, such as between 4 and 8 μm,
wherein the average powder particle size is calculated based on the powder particle number distribution;
c. an average convexity of at least 0.8, such as at least 0.85, such as at least 0.9, such as at least 0.92, such as at least 0.94, such as at least 0.96, such as at least 0.97,
wherein the average convexity is calculated based on the powder particle number distribution or based on the powder particle volume distribution;
d. an average elongation of between 0.4 and 0.7, such as between 0.45 and 0.65,
wherein the average elongation is calculated based on the powder particle volume distribution;
e. an average elongation of between 0.1 and 0.4, such as between 0.2 and 0.3,
wherein the average elongation is calculated based on the powder particle number distribution;
f. an average circularity of at least 0.8, such as at least 0.85, such as at least 0.9,
wherein the average circularity is calculated based on the powder particle volume distribution; and/or
g. an average circularity of at least 0.7, such as at least 0.75, such as at least 0.8,
wherein the average circularity is calculated based on the powder particle number distribution.

8. The BC powder according to any one of the preceding claims, wherein the BC powder comprises:
a. powder particles with a smooth surface; and/or
b. elliptical-shapes powder particles.

9. The BC powder according to any one of the preceding claims, wherein the BC powder is re-dispersible in aqueous media by zero shear or low shear mixing.

10. The BC powder according to any one of the preceding claims, wherein the BC powder is re-dispersible in aqueous media by mixing with a deflocculator, such as a deflocculator mixer, for example a deflocculator turbine.

11. The BC powder according to any one of the preceding claims, wherein the BC powder is re-dispersible in aqueous media by mixing at between 300 rpm and 20,000 rpm, such as between 500 rpm and 5000 rpm, such as between 600 rpm and 10,000 rpm,
optionally by mixing for between 30 seconds and 25 minutes, such as by mixing for between 1 and 20 minutes.

12. The BC powder according to any one of the preceding claims, wherein the rhamnogalacturonan (RG): BC ratio is at most 1:1, such as at most 0.5:1, such as at most 0.1:1, such as at most 0.01:1, such as at most 0.005:1.

13. The BC powder according to any one of the preceding claims, wherein the HG is comprised in pectin.

14. The BC powder according to any one of the preceding claims, wherein the BC powder has a recovery of viscosity of at least 40% upon redispersion, such as at least 50%, such as at least 60%, such as at least 70%, such as at least 80%, such as at least 90%, such as a recovery of viscosity of 100% upon redispersion,
wherein the recovery of viscosity upon redispersion is relative to the viscosity of a corresponding never-dried BC suspension having the same concentration of BC, and wherein the recovery of viscosity is calculated using the formula:
$$Viscosity recovery ( \% ) = 100 * \frac{Viscosity ( at 1 s - 1 ) of redispersed BC powder}{Viscosity ( at 1 s - 1 ) of never dried BC suspension}$$

15. The BC powder according to any one of the preceding claims, wherein the BC powder has a recovery of water holding capacity of at least 50% upon redispersion, such as at least 60%, such as at least 70%, such as at least 80%, such as at least 90%, such as a recovery of water holding capacity of 100% upon redispersion,
wherein the recovery of water holding capacity is relative to the viscosity of a corresponding never-dried BC suspension, and wherein the recovery of viscosity is calculated using the formula:
$$Water holding capacity revovery ( \% ) = 100 * \frac{Water holding capacity of redispersed BC powder}{Water holding capacity of dried BC suspension}$$

16. A method for producing a BC powder, said method comprising providing a BC suspension and:
b. processing said BC suspension;
c. recovering said BC suspension;
d. mixing said BC suspension with HG, thereby obtaining a BC-HG suspension;
e. drying the BC-HG suspension;
thereby obtaining a BC powder
wherein the ratio of HG: BC of said BC powder is at least 0.02:1 (wt:wt).

17. A method for producing a BC powder, said method comprising the steps of:
a. incubating a cellulose-producing bacteria in a culture medium, thereby obtaining a fermentation broth comprising a BC suspension;
b. processing said BC suspension;
c. recovering said BC suspension;
d. mixing said BC suspension with HG, thereby obtaining a BC-HG suspension;
e. drying the BC-HG suspension;
thereby obtaining a BC powder,
wherein the ratio of HG: BC of said BC powder is at least 0.02:1 (wt:wt).

18. The method according to any one of claims 16 to 17, wherein the BC powder is as defined in any one of claims 1 to 27.

19. The method according to any one of claims 17 to 18, wherein the cellulose-producing bacteria is of a genus selected from the group consisting of Acetobacter, Gluconacetobacter, Komagataeibacter and Gluconobacter.

20. The method according to any one of claims 17 to 19, wherein the cellulose-producing bacteria is of a species selected from the group consisting of Acetobacter xylinus, Acetobacter xylinum, Acetobacter hansenii, Acetobacter pasteurianus, Gluconacetobacter xylinus, Gluconobacter oxydans, Komagataeibacter rhaeticus and Komagataeibacter xylinus, preferably wherein the cellulose-producing bacteria is Komagataeibacter xylinus.

21. The method according to any one of claims 17 to 20, wherein the incubation of step a. takes place in an agitated reactor, such as in a stirred tank reactor.

22. The method according to any one of claims 16 to 21, wherein step b. comprises or consists of:
i. washing the recovered BC suspension with water;
ii. treating the BC suspension with an alkali, preferably with sodium hydroxide;
iii. neutralizing the BC suspension with acid; and/or
iv. separating the BC from the fermentation broth, optionally by filtration.

23. The method according to any one of claims 16 to 22, wherein step e. comprises or consists of spray drying or oven drying, preferably wherein step e. comprises spray drying.

24. A BC suspension comprising the BC powder according to any one of claims 1 to 15 resuspended in an aqueous solution.

25. The BC suspension according to claim 24, wherein said BC suspension has a viscosity of at least 1000 mPas, such as at least 1500 mPas, such as at least 2000 mPas, such as at least 2500 mPas at a concentration of 0.5 wt % BC powder,
wherein the viscosity is measured at a shear rate of 1 s⁻¹ at 25° C.

26. The BC suspension according to any one of claims 24 to 25, wherein said BC suspension has a viscosity of at least 6000 mPas, such as at least 8000 mPas, such as at least 10,000 mPas at a concentration of 1.0 wt % BC powder, wherein the viscosity is measured at a shear rate of 1 s⁻¹ at 25° C.

27. The BC suspension according to any one of claims 24 to 26, wherein said BC suspension is capable of stabilizing particles.

28. The BC suspension according to claim 27, wherein said particles have a size of between 0.1 mm and 5 mm in diameter, such as between 0.25 mm and 0.65 m, such as between 1 mm and 2 mm, such as between 1 mm and 1.5 mm, such as between 0.25 mm and 2 mm, such as between 0.1 mm and 4 mm, such as between 0.1 mm and 3 mm.

29. The BC suspension according to any one of claims 24 to 28, wherein said particles are selected from the group consisting of pearlescents; decorative beads; fragrance beads; gelatine beads; jojoba beads; polyethylene beads;
charcoal particles; apricot kernel particles; colorant particles; pigments; air bubbles; insoluble enzymes; encapsulated actives such as moisturizers or exfoliating agents such as alpha-hydroxy acids and glycolic acid; inorganic minerals; kaolin; zeolites; TiO2; ZnO2; vitamins such as vitamin A or vitamin E;
oil droplets; oil suspensions; and oil emulsions,
optionally wherein said particles comprise an active ingredient such as:
i. a vitamin, preferably vitamin A or vitamin E;
ii. retinol, or
iii. an active ingredient which is not soluble in water but is suspendable in water or oil.

30. The BC suspension according to any one of claims 24 to 29, wherein said BC suspension:
a. is stable;
b. does not clump;
c. does not flocculate;
d. does not exhibit water-cellulose separation;
e. is capable of stabilizing particles; and/or
f. maintains its viscosity;
for at least 6 months of storage, such as for at least 9 months of storage, such as for at least 1 year of storage;
at a pH of between 2 and 12, such as at a pH of between 3 and 11;
in the presence of a surfactant;
in the presence of a salt;
in the presence of one or more compounds selected from the group consisting of buffer, particles, emulsifier, thickener, stabilizer and preservative.

31. The BC suspension according to claim 30, wherein
a. the surfactant is a cationic surfactant, a zwitterionic surfactant, an anionic surfactant, an amphoteric surfactant and/or a nonionic surfactant,
optionally wherein the surfactant is selected from the group consisting of cocoamidpropyl betaine (CAPB), sodium dodecyl sulfate (SDS), sodium laureth sulfate (SLS), lauryl glucoside and surfactants comprising quaternary ammonium cation (QUAT), decyl glucose, polyquaternium-7 (QUAT 7), behentrimonium chloride, glyceryl stearate, sodium cetearyl sulphate, glyceryl caprylate, sorbitan laurate/C18-C20 and glycol isostearate,
further optionally wherein the concentration of surfactant is at least 5 wt %, such as at least 10 wt %, such as at least 15 wt %, such as at least 20 wt %;
b. the salt is a sodium salt or a calcium salt,
optionally wherein the salt is selected from the group consisting of sodium chloride, sodium citrate, sodium dihydrogen phosphate and sodium dihydrogen phosphate,
further optionally wherein the concentration of salt is at least 5 wt %, such as at least 10 wt %, such as at least 15 wt %.

32. The BC suspension according to any one of claims 26 to 31, wherein the aqueous solution is water.

33. The BC suspension according to any one of claims 26 to 32, wherein said BC suspension comprises at least 0.02 wt %, such as at least 0.04 wt %, such as at least 0.05 wt %, such as at least 0.1 wt %, such as at least 0.5 wt % BC powder.

34. The BC suspension according to any one of claims 26 to 33, wherein said BC suspension comprises between 0.02 and 3 wt % BC powder, such as between 0.02 and 2.4 wt %, such as between 0.02 and 2 wt %, such as between 0.03 and 2.8 wt %, such as between 0.03 and 2.4 wt %, such as between 0.03 and 2 wt %, such as between 0.04 and 2.8 wt %, such as between 0.04 and 2.4 wt %, such as between 0.04 and 2 wt %, such as between 0.05 and 1.5 wt %, such as between 0.04 and 1.2 wt % BC powder.

35. The BC suspension according to any one of claims 26 to 34, wherein said BC suspension has a water holding capacity of at least 100 g water per g BC (g/g), such as at least 120 g/g, such as at least 130 g/g, such as at least 140 g/g, such as at least 150 g/g.

36. A method for preparing a BC suspension, said method comprising mixing the BC powder of any one of claims 1 to 15 with an aqueous solution, thereby obtaining a BC suspension.

37. The method according to claim 36, wherein the BC suspension is as defined in any one of claims 24 to 35.

38. The method according to any one of claims 36 to 37, the method comprising or consisting of the steps of:
i. performing the method according to any one of claims 16 to 23, thereby obtaining the BC powder; and
ii. mixing the BC powder with an aqueous solution, thereby obtaining the BC suspension.

39. Use of the BC powder according to any one of claims 1 to 15 or the BC suspension according to any one of claims 24 to 35 as a thickener, stabilizer, emulsifier, co-emulsifier, rheology modifier, film-forming agent, sensorial enhancer, SPF-boosting agent, anti-wrinkle agent and/or as a reinforcer material

40. A composition comprising:
a. the BC powder according to any one of claims 1 to 15; or
b. the BC suspension according to any one of claims 24 to 35.

41. The composition according to claim 40, wherein the concentration of BC powder in said composition is between 0.01 and 3 wt %, such as between 0.02 and 3 wt %, such as between 0.03 and 2.5 wt %, such as between 0.04 and 2 wt %.

42. The composition according to any one of claims 40 to 41, wherein said composition further comprises one or more ingredients selected from the group consisting of proteins, vitamins, peptides, beads, salts, oils, particles, humectants, pH adjusters, pH buffers, preservatives, silicones, antioxidants, disinfectants, antimicrobials, emollients, chelating agents, colorants, fragrances, solvents and surfactants.

43. The composition according to claim 42, wherein:
a. the surfactant is as defined in claim 31;
b. the salt is as defined in claim 31;
c. the particle is as defined in any one of claims 28 to 29; and/or
d. the oil is paraffin and/or jojoba oil.

44. The composition according to any one of claims 40 to 43, wherein the composition:
a. is stable;
b. does not clump;
c. does not flocculate; and/or
d. does not exhibit water-cellulose separation,
after at least 3 months of storage, such as after at least 4 months of storage, such as after at least 6 months of storage, such as after at least 1 year of storage.

45. The composition according to any one of claims 40 to 44, wherein said composition does not comprise any additional thickener, stabilizer and/or rheology modifier,
optionally wherein said composition does not comprise any additional polymeric thickener, such as for example a natural polymer, such as for example carboxymethyl cellulose and/or natural gums.

46. The composition according to any one of claims 40 to 45, wherein the composition is aqueous.

47. The composition according to any one of claims 40 to 46, wherein the composition comprises a polyol, such as glycerol or propane diol.

48. A composition comprising:
a. the BC powder according to any one of claims 1 to 15;
b. water; and
c. a quaternary ammonium compound (QUAT), such as a polyquat;
optionally wherein the concentration of BC powder is between 0.05 and 3 wt %, for example between 0.1 and 1 wt %,
further optionally wherein the concentration of QUAT is between 2 and 20 wt %, such as between 5 and 15 wt %.

49. A composition comprising:
a. 0.01 to 3 wt % of resuspended BC powder, wherein the BC powder is the BC powder according to any one of claims 1 to 15;
b. 60 to 90 wt % water;
c. 1 to 10 wt % glycerine; and
d. 5 to 20 wt % surfactant;
optionally wherein the composition further comprises preservative and/or buffer.

50. A product comprising the BC powder according to any one of claims 1 to 15; the BC suspension according to any one of claims 24 to 35; or the composition according to any one of claims 40 to 49.

51. The product according to claim 50, wherein said product is selected from the group consisting of personal care products, cosmetic products, pharmaceutical products, biomedical products and food products.

52. The product according to claim 51, wherein:
a. the cosmetic product is selected from the group consisting of lipstick, mascara, foundation, highlighter, primer, concealer and nail polish and/or
b. the personal care product is selected from the group consisting of cream, lotion, gel, oil, foam, balm, pomade, moisturizer, serum, soap, detergent and scrub, for example wherein the product is facial cleanser, toothpaste, sunscreen, sunblock, shampoo, hair conditioner, hair oil, body lotion, body wash, shower gel, lip balm, shaving cream, shaving gel, deodorant, hand soap, eye cream, eye serum, face cream, anti-wrinkle cream and hand cream.
